SEQUENCE LISTING (136 sequences)

SEQ ID NO: 1 - DNA, 30 bp, Artificial Sequence
  source: /note=“Description of Artificial Sequence Artificial AS ODN Cx43 oligonucleotide”
gtaattgcggcaagaagaattgtttctgtc

SEQ ID NO: 2 - DNA, 30 bp, Artificial Sequence
  source: /note=“Description of Artificial Sequence Artificial AS ODN Cx43 oligonucleotide”
gtaattgcggcaggaggaattgtttctgtc

SEQ ID NO: 3 - DNA, 30 bp, Artificial Sequence
  /note=“Description of Artificial Sequence Artificial AS ODN Cx43 oligonucleotide”
ggcaagagacaccaaagacactaccagcat

SEQ ID NO: 4 - DNA, 27 bp, Artificial Sequence
  source: /note=“Description of Artificial Sequence Artificial AS ODN Cx26 oligonucleotide”
tcctgagcaatacctaacgaacaaata

SEQ ID NO: 5 - DNA, 20 bp, Artificial Sequence
  source: /note=“Description of Artificial Sequence Artificial AS ODN Cx37 oligonucleotide”
catctccttggtgctcaacc

SEQ ID NO: 6 - DNA, 20 bp, Artificial Sequence
  source: /note=“Description of Artificial Sequence Artificial AS ODN Cx37 oligonucleotide”
ctgaagtcgacttggcttgg

SEQ ID NO: 7 - DNA, 21 bp, Artificial Sequence
  source: /note=“Description of Artificial Sequence Artificial AS ODN Cx30 oligonucleotide”
ctcagatagtggccagaatgc

SEQ ID NO: 8 - DNA, 20 bp, Artificial Sequence
  source: /note=“Description of Artificial Sequence Artificial AS ODN Cx30 oligonucleotide”
ttgtccaggtgactccaagg

SEQ ID NO: 9 - DNA, 25 bp, Artificial Sequence
  source: /note=“Description of Artificial Sequence Artificial AS ODN Cx31.1 oligonucleotide”
cgtccgagcccagaaagatgaggtc

SEQ ID NO: 10 - DNA, 19 bp, Artificial Sequence
  source: /note=“Description of Artificial Sequence Artificial AS ODN Cx31.1 oligonucleotide”
agaggcgcacgtgagacac

SEQ ID NO: 11 - DNA, 19 bp, Artificial Sequence
  source: /note=“Description of Artificial Sequence Artificial AS ODN Cx31.1 oligonucleotide”
tgaagacaatgaagatgtt

SEQ ID NO: 12 - DNA, 25 bp, Artificial Sequence
  source: /note=“Description of Artificial Sequence Artificial AS ODN Cx32 oligonucleotide”
tttcttttctatgtgctgttggtga

SEQ ID NO: 13 - protein, 396 aa, Homo sapiens
MSWSFLTRLLEEIHNHSTFVGKIWLTVLIVFRIVLTAVGGESIYYDEQSKFVCNTEQPGCENVCYDAFAPLSHVRFWVFQIILVATPSVMYLGYAIHKIAKMEHGEADKKAARSKPYAMRWKQHRALEETEEDNEEDPMMYPEMELESDKENKEQSQPKPKHDGRRRIREDGLMKIYVLQLLARTVFEVGFLIGQYFLYGFQVHPFYVCSRLPCPHKIDCFISRPTEKTIFLLIMYGVTGLCLLLNIWEMLHLGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKVGSKAGSNKSTASSKSGDGKNSVWI

SEQ ID NO: 14 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 15 - protein, 11 aa, Artificial Sequence
  source: /note=“Description of Artificial Sequence Artificial Cx43 Cx/anti-Cx P peptide”
FEVAFLLIQWI

SEQ ID NO: 16 - protein, 11 aa, Artificial Sequence
  source: /note=“Description of Artificial Sequence Artificial Cx43 Cx/anti-Cx P peptide”
LLIQWYIGFSL

SEQ ID NO: 17 - protein, 16 aa, Artificial Sequence
  source: /note=“Description of Artificial Sequence Artificial Cx43 Cx/anti-Cx P peptide”
SLSAVYTCKRDPCPHQ

SEQ ID NO: 18 - protein, 12 aa, Artificial Sequence
  source: /note=“Description of Artificial Sequence Artificial Cx43 Cx/anti-Cx P peptide”
VDCFLSRPTEKT

SEQ ID NO: 19 - protein, 11 aa, Artificial Sequence
  source: /note=“Description of Artificial Sequence Artificial Cx43 Cx/anti-Cx P peptide”
SRPTEKTIFII

SEQ ID NO: 20 - protein, 13 aa, Artificial Sequence
  source: /note=“Description of Artificial Sequence Artificial Cx43 Cx/anti-Cx P peptide”
LGTAVESAWGDEQ

SEQ ID NO: 21 - protein, 12 aa, Artificial Sequence
  source: /note=“Description of Artificial Sequence Artificial Cx43 Cx/anti-Cx P peptide”
QSAFRCNTQQPG

SEQ ID NO: 22 - protein, 12 aa, Artificial Sequence
  source: /note=“Description of Artificial Sequence Artificial Cx43 Cx/anti-Cx P peptide”
QQPGCENVCYDK

SEQ ID NO: 23 - protein, 13 aa, Artificial Sequence
  source: /note=“Description of Artificial Sequence Artificial Cx43 Cx/anti-Cx P peptide”
VCYDKSFPISHVR

SEQ ID NO: 24 - protein, 38 aa, Artificial Sequence
  source: /note=“Description of Artificial Sequence Artificial Cx32 Cx/anti-Cx P peptide”
AAESVWGDEIKSSFICNTLQPGCNSVCYDHFFPISHVR

SEQ ID NO: 25 - protein, 12 aa, Artificial Sequence
  source: /note=“Description of Artificial Sequence Artificial Cx32 Cx/anti-Cx P peptide”
ESVWGDEKSSFI

SEQ ID NO: 26 - protein, 12 aa, Artificial Sequence
  source: /note=“Description of Artificial Sequence Artificial Cx32 Cx/anti-Cx P peptide”
ICNTLQPGCNSV

SEQ ID NO: 27 - protein, 12 aa, Artificial Sequence
  source: /note=“Description of Artificial Sequence Artificial Cx32 Cx/anti-Cx P peptide”
SVCYDHFFPISH

SEQ ID NO: 28 - protein, 25 aa, Artificial Sequence
  source: /note=“Description of Artificial Sequence Artificial Cx32 Cx/anti-Cx P peptide”
RLVKCEAFPCPNTVDCFVSRPTEKT

SEQ ID NO: 29 - protein, 12 aa, Artificial Sequence
  source: /note=“Description of Artificial Sequence Artificial Cx32 Cx/anti-Cx P peptide”
VKCEAFPCPNTV

SEQ ID NO: 30 - protein, 12 aa, Artificial Sequence
  source: /note=“Description of Artificial Sequence Artificial Cx32 Cx/anti-Cx P peptide”
VDCFVSRPTEKT

SEQ ID NO: 31 - protein, 13 aa, Artificial Sequence
  source: /note=“Description of Artificial Sequence Artificial Cx32 Cx/anti-Cx P peptide”
VCYDHFFPISHVR

SEQ ID NO: 32 - protein, 18 aa, Artificial Sequence
  source: /note=“Description of Artificial Sequence Artificial Cx32 Cx/anti-Cx P peptide”
VWGDEKSSFICNTLQPGY

SEQ ID NO: 33 - protein, 15 aa, Artificial Sequence
  source: /note=“Description of Artificial Sequence Artificial Cx32 Cx/anti-Cx P peptide”
DEKSSFICNTLQPGY

SEQ ID NO: 34 - protein, 11 aa, Artificial Sequence
  source: /note=“Description of Artificial Sequence Artificial Cx32 Cx/anti-Cx P peptide”
SRPTEKTVFTV

SEQ ID NO: 35 - protein, 7 aa, Artificial Sequence
  source: /note=“Description of Artificial Sequence Artificial Cx32/Cx43 Cx/anti-Cx P peptide”
SRPTEKT

SEQ ID NO: 36 - protein, 12 aa, Artificial Sequence
  source: /note=“Description of Artificial Sequence Artificial Cx32 Cx/anti-Cx P peptide”
ICNTLQPGCNSV

SEQ ID NO: 37 - protein, 16 aa, Artificial Sequence
  source: /note=“Description of Artificial Sequence Artificial Cx40 Cx/anti-Cx P peptide”
FLDTLHVCRRSPCPHP

SEQ ID NO: 38 - protein, 18 aa, Artificial Sequence
  source: /note=“Description of Artificial Sequence Artificial Cx43 Cx/anti-Cx P peptide”
KRDPCHQVDCFLSRPTEK

SEQ ID NO: 39 - protein, 35 aa, Artificial Sequence
  source: /note=“Description of Artificial Sequence Artificial huCx26 EC domain polypeptide”
KEVWGDEQADFVCNTLQPGCKNVCYDHYFPISHIR

SEQ ID NO: 40 - protein, 35 aa, Artificial Sequence
  source: /note=“Description of Artificial Sequence Artificial huCx30 EC domain polypeptide”
QEVWGDEQEDFVCNTLQPGCKNVCYDHFFPVSHIR

SEQ ID NO: 41 - protein, 35 aa, Artificial Sequence
  source: /note=“Description of Artificial Sequence Artificial huCx30.3 EC domain polypeptide”
EEVWDDEQKDFVCNTKQPGCPNVCYDEFFPVSHVR

SEQ ID NO: 42 - protein, 35 aa, Artificial Sequence
  source: /note=“Description of Artificial Sequence Artificial huCx31 EC domain polypeptide”
ERVWGDEQKDFDCNTKQPGCTNVCYDNYFPISNIR

SEQ ID NO: 43 - protein, 35 aa, Artificial Sequence
  source: /note=“Description of Artificial Sequence Artificial huCx31.1 EC domain polypeptide”
ERVWSDDHKDFDCNTRQPGCSNVCFDEFFPVSHVR

SEQ ID NO: 44 - protein, 35 aa, Artificial Sequence
  source: /note=“Description of Artificial Sequence Artificial huCx32 EC domain polypeptide”
ESVWGDEKSSFICNTLQPGCNSVCYDQFFPISHVR

SEQ ID NO: 45 - protein, 35 aa, Artificial Sequence
  source: /note=“Description of Artificial Sequence Artificial huCx36 EC domain polypeptide”
ESVWGDEQSDFECNTAQPGCTNVCYDQAFPISHIR

SEQ ID NO: 46 - protein, 35 aa, Artificial Sequence; identical to SEQ ID NO: 45
  source: /note=“Description of Artificial Sequence Artificial huCx37 EC domain polypeptide”

SEQ ID NO: 47 - protein, 35 aa, Artificial Sequence
  source: /note=“Description of Artificial Sequence Artificial huCx40.1 EC domain polypeptide”
RPVYQDEQERFVCNTLQPGCANVCYDVFSPVSHLR

SEQ ID NO: 48 - protein, 35 aa, Artificial Sequence
  source: /note=“Description of Artificial Sequence Artificial huCx43 EC domain polypeptide”
ESAWGDEQSAFRCNTQQPGCENVCYDKSFPISHVR

SEQ ID NO: 49 - protein, 35 aa, Artificial Sequence
  source: /note=“Description of Artificial Sequence Artificial huCx46 EC domain polypeptide”
EDVWGDEQSDFTCNTQQPGCBNVCYBRAFPISHIR

SEQ ID NO: 50 - protein, 35 aa, Artificial Sequence
  source: /note=“Description of Artificial Sequence Artificial huCx46.6 EC domain polypeptide”
EAIYSDEQAKFTCNTRQPGCDNVCYDAFAPLSHVR

SEQ ID NO: 51 - protein, 35 aa, Artificial Sequence
  source: /note=“Description of Artificial Sequence Artificial huCx40 EC domain polypeptide”
ESSWGDEQADFRCDTIQPGCQNVCTDQAFPISHIR

SEQ ID NO: 52 - protein, 36 aa, Artificial Sequence
  source: /note=“Description of Artificial Sequence Artificial huCx45 EC domain polypeptide”
GESIYYDEQSKFVCNTEQPGCENVCYDAFAPLSHVR

SEQ ID NO: 53 - protein, 39 aa, Artificial Sequence
  source: /note=“Description of Artificial Sequence Artificial huCx26 EC domain polypeptide”
MYVFYVMYDGFSMQRLVKCNAWPCPNTVDCFVSRPTEKT

SEQ ID NO: 54 - protein, 39 aa, Artificial Sequence
  source: /note=“Description of Artificial Sequence Artificial huCx30 EC domain polypeptide”
MYVFYFLYNGYHLPWVLKCGIDPCPNLVDCFISRPTEKT

SEQ ID NO: 55 - protein, 39 aa, Artificial Sequence
  source: /note=“Description of Artificial Sequence Artificial huCx30.3 EC domain polypeptide”
LYIFHRLYKDYDMPRVVACSVEPCPHTVDCYISRPTEKK

SEQ ID NO: 56 - protein, 40 aa, Artificial Sequence
  source: /note=“Description of Artificial Sequence Artificial huCx31 EC domain polypeptide”
LYLLHTLWHGFNMPRLVQCANVAPCPNIVDCYIARPTEKK

SEQ ID NO: 57 - protein, 39 aa, Artificial Sequence
  source: /note=“Description of Artificial Sequence Artificial huCx31.1 EC domain polypeptide”
LYVFHSFYPKYILPPVVKCHADPCPNIVDCFISKPSEKN

SEQ ID NO: 58 - protein, 39 aa, Artificial Sequence
  source: /note=“Description of Artificial Sequence Artificial huCx32 EC domain polypeptide”
MYVFYLLYPGYAMVRLVKCDVYPCPNTVDCFVSRPTEKT

SEQ ID NO: 59 - protein, 32 aa, Artificial Sequence
  source: /note=“Description of Artificial Sequence Artificial huCx36 EC domain polypeptide”
LYGWTMEPVFVCQRAPCPYLVDCFVSRPTEKT

SEQ ID NO: 60 - protein, 32 aa, Artificial Sequence; identical to SEQ ID NO: 59
  source: /note=“Description of Artificial Sequence Artificial huCx37 EC domain polypeptide”

SEQ ID NO: 61 - protein, 38 aa, Artificial Sequence
  source: /note=“Description of Artificial Sequence Artificial huCx40.1 EC domain polypeptide”
GALHYFLFGFLAPKKFPCTRPPCTGVVDCYVSRPTSKS

SEQ ID NO: 62 - protein, 38 aa, Artificial Sequence
  source: /note=“Description of Artificial Sequence Artificial huCx43 EC domain polypeptide”
LLIQWYIYGFSLSAVYTCKRDPCPHQVDCFLSRPTEKT

SEQ ID NO: 63 - protein, 38 aa, Artificial Sequence
  source: /note=“Description of Artificial Sequence Artificial huCx46 EC domain polypeptide”
IAGQYFLYGFELKPLYRCDRWPCPNTVDCFISRPTEKT

SEQ ID NO: 64 - protein, 38 aa, Artificial Sequence
  source: /note=“Description of Artificial Sequence Artificial huCx46.6 EC domain polypeptide”
LVGQYLLYGFEVRPFFPCSRQPCPHVVDCFVSRPTEKT

SEQ ID NO: 65 - protein, 38 aa, Artificial Sequence
  source: /note=“Description of Artificial Sequence Artificial huCx40 EC domain polypeptide”
IVGQYFIYGIFLTTLHVCRRSPCPHPVNCYVSRPTEKN

SEQ ID NO: 66 - protein, 38 aa, Artificial Sequence
  source: /note=“Description of Artificial Sequence Artificial huCx45 EC domain polypeptide”
LIGQYFLYGFQVHPFYVCSRLPCHPKIDCFISRPTEKT

SEQ ID NO: 67 - protein, 42 aa, Artificial Sequence
  source: /note=“Description of Artificial Sequence Artificial huCx43 EC domain polypeptide”
LLIQWYIYGFSLSAVYTCKRDPCPHQVDCFLSRPTEKTIFII

SEQ ID NO: 68 - protein, 43 aa, Artificial Sequence
  source: /note=“Description of Artificial Sequence Artificial huCx26 EC domain polypeptide”
MYVFYVMYDGFSMQRLVKCNAWPCPNTVDCFVSRPTEKTVFTV

SEQ ID NO: 69 - protein, 42 aa, Artificial Sequence
  source: /note=“Description of Artificial Sequence Artificial huCx30 EC domain polypeptide”
YVFYFLYNGYHLPWVLKCGIDPCPNLVDCFISRPTEKTVFTI

SEQ ID NO: 70 - protein, 43 aa, Artificial Sequence
  source: /note=“Description of Artificial Sequence Artificial huCx30.3 EC domain polypeptide”
LYIFHRLYKDYDMPRVVACSVEPCPHTVDCYISRPTEKKVFTY

SEQ ID NO: 71 - protein, 42 aa, Artificial Sequence
  source: /note=“Description of Artificial Sequence Artificial huCx31 EC domain polypeptide”
LYLLHTLWHGFNMPRLVQCANVAPCPNIVDCYIARPTEKKTY

SEQ ID NO: 72 - protein, 43 aa, Artificial Sequence
  source: /note=“Description of Artificial Sequence Artificial huCx31.1 EC domain polypeptide”
LYVFHSFYPKYILPPVVKCHADPCPNIVDCFISKPSEKNIFTL

SEQ ID NO: 73 - protein, 43 aa, Artificial Sequence
  source: /note=“Description of Artificial Sequence Artificial huCx32 EC domain polypeptide”
MYVFYLLYPGYAMVRLVKCDVYPCPNTVDCFVSRPTEKTVFTV

SEQ ID NO: 74 - protein, 36 aa, Artificial Sequence
  source: /note=“Description of Artificial Sequence Artificial huCx36 EC domain polypeptide”
LYGWTMEPVFVCQRAPCPYLVDCFVSRPTEKTIFII

SEQ ID NO: 75 - protein, 36 aa, Artificial Sequence; identical to SEQ ID NO: 74
  source: /note=“Description of Artificial Sequence Artificial huCx37 EC domain polypeptide”

SEQ ID NO: 76 - protein, 42 aa, Artificial Sequence
  source: /note=“Description of Artificial Sequence Artificial huCx40.1 EC domain polypeptide”
GALHYFLFGFLAPKKFPCTRPPCTGVVDCYVSRPTEKSLLML

SEQ ID NO: 77 - protein, 42 aa, Artificial Sequence
  source: /note=“Description of Artificial Sequence Artificial huCx46 EC domain polypeptide”
IAGQYFLYGFELKPLYRCDRWPCPNTVDCFISRPTEKTIFII

SEQ ID NO: 78 - protein, 42 aa, Artificial Sequence
  source: /note=“Description of Artificial Sequence Artificial huCx46.1 EC domain polypeptide”
LVGQYLLYGFEVRPFFPCSRQPCPHVVDCFVSRPTEKTVFLL

SEQ ID NO: 79 - protein, 41 aa, Artificial Sequence
  source: /note=“Description of Artificial Sequence Artificial huCx40 EC domain polypeptide”
IVGQYFIYGIFLTTLHVCRRSPCPHPVNCYSRPTEKNVFIV

SEQ ID NO: 80 - protein, 42 aa, Artificial Sequence
  source: /note=“Description of Artificial Sequence Artificial huCx45 EC domain polypeptide”
LIGQYFLYGFQVHPFYVCSRLPCHPKIDCFISRPTEKTIFLL

SEQ ID NO: 81 - protein, 45 aa, Artificial Sequence
  source: /note=“Description of Artificial Sequence Artificial Cx40 Cx/anti-Cx P polypeptide”
LGTAAESSWGDEQADFRCDTIQPGCQNVCTDQAFPISHIRFWVLQ

SEQ ID NO: 82 - protein, 14 aa, Artificial Sequence
  source: /note=“Description of Artificial Sequence Artificial Cx40 Cx/anti-Cx P peptide”
LGTAAESSWGDEQA

SEQ ID NO: 83 - protein, 13 aa, Artificial Sequence
  source: /note=“Description of Artificial Sequence Artificial Cx40 Cx/anti-Cx P peptide”
DEQADFRCDTIQP

SEQ ID NO: 84 - protein, 13 aa, Artificial Sequence
  source: /note=“Description of Artificial Sequence Artificial Cx40 Cx/anti-Cx P peptide”
TIQPGCQNVCTDQ

SEQ ID NO: 85 - protein, 13 aa, Artificial Sequence
  source: /note=“Description of Artificial Sequence Artificial Cx40 Cx/anti-Cx P peptide”
VCTDQAFPISHIR

SEQ ID NO: 86 - protein, 13 aa, Artificial Sequence
  source: /note=“Description of Artificial Sequence Artificial Cx40 Cx/anti-Cx P peptide”
AFPISHIRFWVLQ

SEQ ID NO: 87 - protein, 47 aa, Artificial Sequence
  source: /note=“Description of Artificial Sequence Artificial Cx40 Cx/anti-Cx P polypeptide”
MEVGFIVGQYFIYGIFLTTLHVCRRSPCPHPVNCYVSRPTEKNVFIV

SEQ ID NO: 88 - protein, 11 aa, Artificial Sequence
  source: /note=“Description of Artificial Sequence Artificial Cx40 Cx/anti-Cx P peptide”
MEVGFIVGQYF

SEQ ID NO: 89 - protein, 12 aa, Artificial Sequence
  source: /note=“Description of Artificial Sequence Artificial Cx40 Cx/anti-Cx P peptide”
IVGQYFIYGIFL

SEQ ID NO: 90 - protein, 14 aa, Artificial Sequence
  source: /note=“Description of Artificial Sequence Artificial Cx40 Cx/anti-Cx P peptide”
GIFLTTLHVCRRSP

SEQ ID NO: 91 - protein, 12 aa, Artificial Sequence
  source: /note=“Description of Artificial Sequence Artificial Cx40 Cx/anti-Cx P peptide”
RRSPCPHPVNCY

SEQ ID NO: 92 - protein, 12 aa, Artificial Sequence
  source: /note=“Description of Artificial Sequence Artificial Cx40 Cx/anti-Cx P peptide”
VNCYVSRPTEKN

SEQ ID NO: 93 - protein, 11 aa, Artificial Sequence
  source: /note=“Description of Artificial Sequence Artificial Cx40 Cx/anti-Cx P peptide”
SRPTEKNVFIV

SEQ ID NO: 94 - protein, 46 aa, Artificial Sequence
  source: /note=“Description of Artificial Sequence Artificial Cx45 Cx/anti-Cx P polypeptide”
LTAVGGESIYYDEQSKFVCNTEQPGCENVCYDAFAPLSHVRFWVFQ

SEQ ID NO: 95 - protein, 15 aa, Artificial Sequence
  source: /note=“Description of Artificial Sequence Artificial Cx45 Cx/anti-Cx P peptide”
LTAVGGESIYYDEQS

SEQ ID NO: 96 - protein, 13 aa, Artificial Sequence
  source: /note=“Description of Artificial Sequence Artificial Cx45 Cx/anti-Cx P peptide”
DEQSKFVCNTEQP

SEQ ID NO: 97 - protein, 13 aa, Artificial Sequence
  source: /note=“Description of Artificial Sequence Artificial Cx45 Cx/anti-Cx P peptide”
TEQPGCENVCYDA

SEQ ID NO: 98 - protein, 13 aa, Artificial Sequence
  source: /note=“Description of Artificial Sequence Artificial Cx45 Cx/anti-Cx P peptide”
VCYDAFAPLSHVR

SEQ ID NO: 99 - protein, 12 aa, Artificial Sequence
  source: /note=“Description of Artificial Sequence Artificial Cx45 Cx/anti-Cx P peptide”
APLSHVRFWVFQ

SEQ ID NO: 100 - protein, 47 aa, Artificial Sequence
  source: /note=“Description of Artificial Sequence Artificial Cx45 Cx/anti-Cx P polypeptide”
FEVGFLIGQYFLYGFQVHPFYVCSRLPCHPKIDCFISRPTEKTIFLL

SEQ ID NO: 101 - protein, 11 aa, Artificial Sequence
  source: /note=“Description of Artificial Sequence Artificial Cx45 Cx/anti-Cx P peptide”
FEVGFLIGQYF

SEQ ID NO: 102 - protein, 12 aa, Artificial Sequence
  source: /note=“Description of Artificial Sequence Artificial Cx45 Cx/anti-Cx P peptide”
LIGQYFLYGFQV

SEQ ID NO: 103 - protein, 14 aa, Artificial Sequence
  source: /note=“Description of Artificial Sequence Artificial Cx45 Cx/anti-Cx P peptide”
GFQVHPFYVCSRLP

SEQ ID NO: 104 - protein, 12 aa, Artificial Sequence
  source: /note=“Description of Artificial Sequence Artificial Cx45 Cx/anti-Cx P peptide”
SRLPCHPKIDCF

SEQ ID NO: 105 - protein, 12 aa, Artificial Sequence
  source: /note=“Description of Artificial Sequence Artificial Cx45 Cx/anti-Cx P peptide”
IDCFISRPTEKT

SEQ ID NO: 106 - protein, 11 aa, Artificial Sequence
  source: /note=“Description of Artificial Sequence Artificial Cx45 Cx/anti-Cx P peptide”
SRPTEKTIFLL

SEQ ID NO: 107 - protein, 11 aa, Artificial Sequence
  source: /note=“Description of Artificial Sequence Artificial Cx/anti-Cx Control P peptide”
SRGGEKNVFIV

SEQ ID NO: 108 - protein, 10 aa, Artificial Sequence
  source: /note=“Description of Artificial Sequence Artificial Cx45 Cx/anti-Cx P peptide”
YVCSRLPCHP

SEQ ID NO: 109 - protein, 11 aa, Artificial Sequence
  source: /note=“Description of Artificial Sequence Artificial Cx45 Cx/anti-Cx P peptide”
QVHPFYVCSRL

SEQ ID NO: 110 - protein, 13 aa, Artificial Sequence
  source: /note=“Description of Artificial Sequence Artificial Cx45 Cx/anti-Cx P peptide”
FEVGFLIGQYFLY

SEQ ID NO: 111 - protein, 12 aa, Artificial Sequence
  source: /note=“Description of Artificial Sequence Artificial Cx45 Cx/anti-Cx P peptide”
GQYFLYGFQVHP

SEQ ID NO: 112 - protein, 12 aa, Artificial Sequence
  source: /note=“Description of Artificial Sequence Artificial Cx45 Cx/anti-Cx P peptide”
GFQVHPFYVCSR

SEQ ID NO: 113 - protein, 12 aa, Artificial Sequence
  source: /note=“Description of Artificial Sequence Artificial Cx45 Cx/anti-Cx P peptide”
AVGGESIYYDEQ

SEQ ID NO: 114 - protein, 12 aa, Artificial Sequence
  source: /note=“Description of Artificial Sequence Artificial Cx45 Cx/anti-Cx P peptide”
YDEQSKFVCNTE

SEQ ID NO: 115 - protein, 12 aa, Artificial Sequence
  source: /note=“Description of Artificial Sequence Artificial Cx45 Cx/anti-Cx P peptide”
NTEQPGCENVCY

SEQ ID NO: 116 - protein, 12 aa, Artificial Sequence
  source: /note=“Description of Artificial Sequence Artificial Cx45 Cx/anti-Cx P peptide”
CYDAFAPLSHVR

SEQ ID NO: 117 - protein, 12 aa, Artificial Sequence
  source: /note=“Description of Artificial Sequence Artificial Cx45 Cx/anti-Cx P peptide”
FAPLSHVRFWVF

SEQ ID NO: 118 - protein, 5 aa, Artificial Sequence
  source: /note=“Description of Artificial Sequence Artificial Cx45 Cx/anti-Cx P peptide”
LIGQY

SEQ ID NO: 119 - protein, 5 aa, Artificial Sequence
  source: /note=“Description of Artificial Sequence Artificial Cx45 Cx/anti-Cx P peptide”
QVHPF

SEQ ID NO: 120 - protein, 5 aa, Artificial Sequence
  source: /note=“Description of Artificial Sequence Artificial Cx45 Cx/anti-Cx P peptide”
YVCSR

SEQ ID NO: 121 - protein, 5 aa, Artificial Sequence
  source: /note=“Description of Artificial Sequence Artificial Cx45 Cx/anti-Cx P peptide”
SRLPC

SEQ ID NO: 122 - protein, 5 aa, Artificial Sequence
  source: /note=“Description of Artificial Sequence Artificial Cx45 Cx/anti-Cx P peptide”
LPCHP

SEQ ID NO: 123 - protein, 5 aa, Artificial Sequence
  source: /note=“Description of Artificial Sequence Artificial Cx45 Cx/anti-Cx P peptide”
GESIY

SEQ ID NO: 124 - protein, 6 aa, Artificial Sequence
  source: /note=“Description of Artificial Sequence Artificial Cx45 Cx/anti-Cx P peptide”
YDEQSK

SEQ ID NO: 125 - protein, 6 aa, Artificial Sequence
  source: /note=“Description of Artificial Sequence Artificial Cx45 Cx/anti-Cx P peptide”
SKFVCN

SEQ ID NO: 126 - protein, 8 aa, Artificial Sequence
  source: /note=“Description of Artificial Sequence Artificial Cx45 Cx/anti-Cx P peptide”
TEQPGCEN

SEQ ID NO: 127 - protein, 8 aa, Artificial Sequence
  source: /note=“Description of Artificial Sequence Artificial Cx45 Cx/anti-Cx P peptide”
VCYDAFAP

SEQ ID NO: 128 - protein, 10 aa, Artificial Sequence
  source: /note=“Description of Artificial Sequence Artificial Cx45 Cx/anti-Cx P peptide”
LSHVRFWVFQ

SEQ ID NO: 129 - protein, 14 aa, Artificial Sequence
  source: /note=“Description of Artificial Sequence Artificial Cx45 Cx/anti-Cx P peptide”
LIQYFLYGFQVHPF

SEQ ID NO: 130 - protein, 13 aa, Artificial Sequence
  source: /note=“Description of Artificial Sequence Artificial Cx45 Cx/anti-Cx P peptide”
VHPFYCSRLPCHP

SEQ ID NO: 131 - protein, 22 aa, Artificial Sequence
  source: /note=“Description of Artificial Sequence Artificial Cx45 Cx/anti-Cx P peptide”
VGGESIYYDEQSKFVCNTEQPG

SEQ ID NO: 132 - protein, 22 aa, Artificial Sequence
  source: /note=“Description of Artificial Sequence Artificial Cx45 Cx/anti-Cx P peptide”
TEQPGCENVCYDAFAPLSHVRF

SEQ ID NO: 133 - protein, 14 aa, Artificial Sequence
  source: /note=“Description of Artificial Sequence Artificial Cx45 Cx/anti-Cx P peptide”
AFAPLSHVRFWVFQ

SEQ ID NO: 134 - DNA, 1314 bp, Homo sapiens
ggcttttagcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 135 - DNA, 1149 bp, Homo sapiens
atgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 136 - DNA, 30 bp, Artificial Sequence
  source: /note=“Description of Artificial Sequence Artificial Sense Control oligonucleotide”
gacagaaacaattcctcctgccgcatttac

(19) United States
(12) Patent Application Publication
(10) Pub. No.: US 2010/0279921 A1
(43) Pub. Date: Nov. 4, 2010

(54) IMPAIRED WOUND HEALING COMPOSITIONS AND TREATMENTS

(75) Inventor: Bradford James Duft, Rancho Santa Fe, CA (US)

Correspondence Address: DUANE MORRIS LLP - San Diego, 101 WEST BROADWAY, SUITE 900, SAN DIEGO, CA, 92101-8285, US

(73) Assignee: CoDa Therapeutics, Inc., San Diego, CA (US)

(21) Appl. No.: 12/747,863

(22) PCT Filed: Dec. 11, 2008
(86) PCT No.: PCT/US08/13656

§ 371 Date: Jun. 11, 2010

Related U.S. Application Data
(60) Provisional application No. 61/007,262, filed on Dec. 11, 2007.

(51) Int. Cl. A61K 31/713; A61K 38/02; A61P 17/02

(52) U.S. Cl. 514/1.2; 514/44 A

(57) ABSTRACT

Methods and compositions comprising combinations and uses of a first anti-connexin agent and a second anti-connexin agent, for example, one or more anti-connexin polynucleotides and one or more anti-connexin peptides or peptidomimetics, are provided for therapeutic use including uses for the promotion and/or improvement of wounds and wound healing and/or tissue repair.

This application is a National Stage Application under 35 U.S.C. §371 of International Application No. PCT/US2008/013656, filed on Dec. 11, 2008 which claims the benefit of priority to U.S. Provisional Application No. 61/007,262 filed on Dec. 11, 2007. The disclosures of both are incorporated herein by reference.

### FIELD

The inventions relate to gap junctions and to wounds and wound healing, in particular to acute wounds and to wounds that do not heal at expected rates, such as delayed-healing wounds, incompletely healing wounds, chronic wounds, and dehiscent wounds.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

In humans and other mammals wound injury triggers an organized complex cascade of cellular and biochemical events that will in most cases result in a healed wound. An ideally healed wound is one that restores normal anatomical structure, function, and appearance on cellular, tissue, organ, and organism levels. Wound healing, whether initiated by trauma, microbes or foreign materials, proceeds via a complex process encompassing a number of overlapping phases, including inflammation, epithelialization, angiogenesis and matrix deposition. Normally, these processes lead to a mature wound and a certain degree of scar formation. Although inflammation and repair mostly occur along a prescribed course, the sensitivity of the process is dependent on the balance of a variety of wound healing molecules, including for example, a network of regulatory cytokines and growth factors.

Gap junctions are cell membrane structures that facilitate direct cell-cell communication. A gap junction channel is formed of two connexons (hemichannels), each composed of six connexin subunits. Each hexameric connexon docks with a connexon in the opposing membrane to form a single gap junction. Gap junction channels are reported to be found throughout the body. Tissue such as the corneal epithelium, for example, has six to eight cell layers, yet expresses different gap junction channels in different layers with connexin 43 in the basal layer and connexin 26 from the basal to middle wing cell layers. In general, connexins are a family of proteins, commonly named according to their molecular weight or classified on a phylogenetic basis into alpha, beta, and gamma subclasses. At least 20 human and 19 murine isoforms have been identified. Different tissues and cell types are reported to have characteristic patterns of connexin protein expression and tissues have been shown to alter connexin protein expression pattern following injury or transplantation (Qui, C. et al., (2003) Current Biology, 13:1967-1703; Brander et al., (2004), J. Invest Dermatol. 122:1310-20).

It has been reported that abnormal connexin function may be linked to certain disease states (e.g. heart diseases) (A. C. de Carvalho, et al., J Cardiovasc Electrophysiol 1994, 5 686). In certain connexin proteins, alterations in the turnover and trafficking properties may be induced by the addition exogenous agents which may affect the level of gap junctional intercellular communication (Darrow, B. J., et al. (1995). Circ Res 76: 381; Lin R, et al. (2001) J Cell Biol 154(4):815). Antisense technology has been proposed for the modulation of the expression for genes implicated in viral, fungal and metabolic diseases. See, for example, U.S. Pat. No. 5,166,195, (oligonucleotide inhibitors of HIV) and U.S. Pat. No. 5,004,810 (oligomers for hybridizing to herpes simplex virus Vmw65 mRNA and inhibiting replication). See also U.S. Pat. No. 7,098,190 issued to Becker and Green (“Formulations comprising antisense nucleotides to connexins”). Peptide inhibitors of gap junctions and hemichannels have also been reported. See for example Berthoud, V. M. et al., Am J. Physiol. Lung Cell Mol. Physiol. 279:L619-L622 (2000); Evans, W. H. and Boitano, S. Biochem. Soc. Trans. 29:606-612, and De Vriese A. S., et al. Kidney Int. 61:177-185 (2001). See also Becker and Green PCT/US06/04131 (“Anti-connexin compounds and uses thereof”).

Despite advances in the understanding of the principles underlying the wound healing process, there remains a significant unmet need in suitable therapeutic options for wound care, including wounds that do not heal at expected rates, such as delayed-healing wounds, incompletely healing wounds, and chronic wounds. Such therapeutics compositions and treatments are described and claimed herein.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The invention generally relates to the use of one or more anti-connexin polynucleotides (for example, connexin inhibitors such as alpha-1 connexin oligodeoxynucleotides) in combination with one or more anti-connexin peptides, peptidomimetics (for example, alpha-1 anti-connexin peptides or peptidomimetics) gap junction closing compounds, hemichannel closing compounds, and connexin carboxy-terminal polypeptides for the treatment of wounds, including acute, delayed healing and chronic wounds.

Compositions and methods of the invention that employ a first anti-connexin agent in combination with a second anti-connexin agent are disclosed and claimed. A first anti-connexin agent may be selected from the group consisting of anti-connexin oligonucleotides, anti-connexin peptides, anti-connexin peptidomimetics, gap junction closing compounds, hemichannel closing compounds, connexin carboxy-terminal polypeptides and other gap junction modulating agents useful for wound healing. A second anti-connexin agent is selected from the above group as modified to subtract the subcategory of anti-connexin agents from which the first anti-connexin agent was selected.

The invention includes a pharmaceutical composition comprising a pharmaceutically acceptable anti-connexin polynucleotide and a pharmaceutically acceptable anti-connexin peptide or peptidomimetic, for wound healing in amounts effective to promote healing or tissue repair in a subject. It also includes a pharmaceutical composition comprising a first anti-connexin agent and a second anti-connexin agent, wherein the first anti-connexin agent is selected from the group consisting of anti-connexin oligonucleotides, anti-connexin peptides, anti-connexin peptidomimetics, gap junction closing compounds, hemichannel closing compounds, and connexin carboxy-terminal polypeptides useful for wound healing, and the second anti-connexin agent is selected from the above group as modified to subtract the subcategory of anti-connexin agents from which the first anti-connexin agent was selected. Such formulations include, for example, topical delivery forms and formulations. Such delivery forms and formulations include those for the treatment of a subject as disclosed herein. Preferred anti-connexin polynucleotides are anti-connexin 43 oligonucleotides (ODN). Preferred peptides or peptidomimetics, are anti-connexin 43 peptides or peptidomimetics, e.g., anti-connexin 43 hemichannel blocking peptides or anti-connexin 43 hemichannel blocking peptidomimetics. Preferred gap junction closing compounds and hemichannel closing compounds are connexin 43 gap junction closing compounds and connexin 43 hemichannel closing compounds. Preferred connexin carboxy-terminal polypeptides are connexin 43 carboxy-terminal polypeptides. Treatment of a subject, e.g., for a wound, with one or more pharmaceutical compositions of the invention, e.g., an anti-connexin ODN and a connexin hemichannel blocking agent, e.g., a peptide or peptidomimetic, or a first anti-connexin agent and a second anti-connexin agent, may comprise their simultaneous, separate, sequential or sustained administration.

The invention includes pharmaceutical compositions comprising (a) an anti-connexin peptide or pepidomimetic and (b) an antisense polynucleotide to the mRNA of a connexin protein. Most preferably, this connexin is connexin 43. The invention also includes pharmaceutical compositions comprising (a) and/or (b) and one or more of a gap junction closing compound, a hemichannel closing compound, and a connexin carboxy-terminal polypeptide useful for wound healing. Most preferably, in the case of gap junction closing compound and hemichannel closing compounds useful for wound healing the gap junction or hemichannel is a connexin 43 gap junction or hemichannel. Most preferably, in the case of connexin carboxy-terminal polypeptides useful for wound healing, the connexin is connexin 43.

Pharmaceutical compositions are also provided in the form of a combined preparation, for example, as an admixture of two or more anti-connexin agents, for example one or more anti-connexin polynucleotides and one or more anti-connexin peptides or peptidomimetics.

The term “a combined preparation” includes a “kit of parts” in the sense that the combination partners as defined above can be dosed independently or by use of different fixed combinations with distinguished amounts of the combination partners (a) and (b), i.e. simultaneously, separately or sequentially, whether in pharmaceutical form or dressing/matrix form or both. The parts of the kit can then, for example, be administered simultaneously or chronologically staggered, that is at different time points and with equal or different time intervals for any part of the kit of parts.

In one embodiment a combined preparation is administered, wherein two or more separate compositions are administered to a subject, wherein the first composition comprises a therapeutically effective amount of a anti-connexin 43 polynucleotide and the second composition comprises a therapeutically effective amount of an anti-connexin 43 peptide or peptidomimetic. In another embodiment a third composition is administered comprising one or more anti-connexin polynucleotides, peptides, or peptidomimetics. The third composition may also comprise one or more gap junction closing compounds, hemichannel closing compounds, or connexin carboxy-terminal polypeptides useful for wound healing.

Pharmaceutical compositions are provided for combined, simultaneous, separate sequential or sustained administration. In one embodiment, a composition comprising one or more anti-connexin polynucleotides is administered at or about the same time as one or more anti-connexin peptides or peptidomimetics. In one embodiment, a composition comprising one or more anti-connexin polynucleotides is administered within at least about thirty minutes of one or more anti-connexin peptides or peptidomimetics. In one embodiment, a composition comprising one or more anti-connexin polynucleotides is administered within at least about one hours of one or more anti-connexin peptides or peptidomimetics. In one embodiment, a composition comprising one or more anti-connexin polynucleotides is administered within at least about twelve hours of one or more anti-connexin peptides or peptidomimetics. In one embodiment, a composition comprising one or more anti-connexin polynucleotides is administered within at least about twenty-four hours of one or more anti-connexin peptides or peptidomimetics. In another embodiment the anti-connexin polynucleotide and anti-connexin peptide or peptidomimetic are administered within about one hour of each other, within about one day of each other, or within about one week of each other. Other embodiments include administration of one or more anti-connexin polynucleotides and/or one or more anti-connexin peptides or peptidomimetics, and one or more gap junction closing compounds useful for wound healing, one or more hemichannel closing compounds useful for wound healing, and/or one or more connexin carboxy-terminal polypeptides useful for wound healing.

In one aspect, the invention includes pharmaceutical compositions, including topical delivery forms and formulations, comprising a pharmaceutically acceptable carrier and therapeutically effective amounts of a first anti-connexin agent and a second anti-connexin agent as described herein, for example, an anti-connexin polynucleotide useful for wound healing and one or more anti-connexin peptides or peptidomimetics useful for wound healing. Examples of anti-connexin polynucleotides include anti-connexin oligodeoxynucleotides (“ODN”), including antisense (including modified and unmodified backbone antisense), RNAi, and siRNA. Suitable anti-connexin peptides include binding peptides. Suitable anti-connexin agents include for example, antisense ODNs, peptides and peptidomimetics against connexins 43, 26, 37, 30, and 31.1 and 32. In certain embodiments, suitable compositions include multiple anti-connexin agents in combination, including for example, connexin 43, 26, 30, and 31.1. Preferred anti-connexin agents, including anti-connexin oligonucleotides and anti-connexin peptides and peptidomimetics, are directed against connexin 43.

The present invention provides for an increase in the rate, extent and/or quality of wound healing through the use of two or more anti-connexin agents administered simulataneously, separate, or sequentially. In a preferred embodiment, the combined use of a first anti-connexin agent and a second anti-connexin agent as described herein, for example, one or more anti-connexin polynucleotides and one or more anti-connexin peptides or peptidomimetics has an additive, synergistic or super-additive effect in the promotion of wound healing. In a preferred embodiment, the administration of a combined preparation will have fewer administration time points and/or increased time intervals between administrations as a result of such combined use. In another preferred embodiment, the combined use of a first anti-connexin agent and a second anti-connexin agent as described herein, for example, one or more anti-connexin polynucleotides and one or more anti-connexin peptides or peptidomimetics, allows a reduced frequency of administration. In another preferred embodiment, the combined use of a first anti-connexin agent and a second anti-connexin agent as described herein, for example, one or more anti-connexin polynucleotides and one or more anti-connexin peptides or peptidomimetics, allows the use of reduced doses of such agents compared to the dose or doses that may be effective when the agent is administered alone.

In another aspect, the invention includes methods for administering a therapeutically effective amount of a first anti-connexin agent and a second anti-connexin agent as described herein, for example, one or more anti-connexin polynucleotides and one or more anti-connexin peptides or peptidomimetics, formulated in a delayed release preparation, a slow release preparation, an extended release preparation, a controlled release preparation, and/or in a repeat action preparation to a subject with a wound, including wounds characterized in whole or in part by delayed or incomplete wound healing.

In certain other aspects, the invention also relates to methods of using such compositions to treat subjects suffering from or at risk for various diseases, disorders, and conditions associated with a wound, including acute and, as well as wounds that do not heal at expected rates, including delayed healing and chronic wounds.

In yet another aspect, the invention includes methods for treating a subject having or suspected of having or predisposed to, or at risk for, any diseases, disorders and/or conditions characterized in whole or in part by a wound or a tissue in need of repair. Such compositions include, for example, topical delivery forms and formulations.

In another aspect, the invention provides method of treatment comprising administering to a subject a pharmaceutical composition of the invention for use in the treatment of a wound, including for example, acute, as well as wounds that do not heal at expected rates, including delayed healing and chronic wounds.

In another aspect, the invention provides a method of treatment comprising administering to a subject in need thereof a composition comprising therapeutically effective amounts of a first anti-connexin agent and a second anti-connexin agent, wherein said first agent is an anti-connexin polynucleotide agent and said second agent is an anti-connexin peptide or peptidomimetic.

In yet another aspect, the invention provides a method of treatment comprising administering to a subject in need thereof a first composition and a second composition, said first composition comprising a therapeutically effective amount of a anti-connexin 43 polynucleotide and said second composition comprising a therapeutically effective amount of an anti-connexin 43 peptide or peptidomimetic. In one embodiment the first composition is administered first. In another embodiment, the second composition is administered first. In a further embodiment, the method, further comprises administration of a third composition, wherein the third wound healing composition comprises an anti-connexin polynucleotide, peptide or peptidomimetic. In one embodiment the third composition is administered first.

In one aspect, the invention provides a method for treating acute wounds, comprising administering to a subject in need thereof a therapeutically effective amount of a pharmaceutical composition comprising a first anti-connexin agent and a second anti-connexin agent as described herein, for example, one or more anti-connexin polynucleotides and one or more anti-connexin peptides or peptidomimetics. In one embodiment, said method comprises administration of two pharmaceutical compositions, the first composition comprising one or more anti-connexin polynucleotides and the second pharmaceutical composition comprising one or more anti-connexin peptides or peptidomimetics. In one embodiment the first composition is administered first. In another embodiment, the second composition is administered first. In a further embodiment, the method, further comprises administration of a third composition, wherein the third wound healing composition comprises an anti-connexin polynucleotide, peptide or peptidomimetic. In one embodiment the third composition is administered first. In one embodiment the third composition is administered first. In one embodiment the pharmaceutical compositions are administered topically.

In one aspect, the invention provides a method for treating chronic wounds, or delayed or slow healing wounds comprising administering to a subject in need thereof a therapeutically effective amount of a pharmaceutical composition comprising a first anti-connexin agent and a second anti-connexin agent as described herein, for example, one or more anti-connexin polynucleotides and one or more anti-connexin peptides or peptidomimetics. In one embodiment, said method comprises administration of two pharmaceutical compositions, the first composition comprising one or more anti-connexin polynucleotides and the second pharmaceutical composition comprising one or more anti-connexin peptides or peptidomimetics. In certain embodiments the chronic wound is a diabetic ulcer, a diabetic foot ulcer, a venous ulcer, a venous stasis ulcer, a pressure ulcer, a decubitus ulcer, a vasculitic ulcer, an arterial ulcer, an infectious ulcer, a burn ulcer, a trauma-induced ulcer, or an ulceration associated with pyoderma gangrenosum. In one embodiment the subject is diabetic. In one embodiment the subject has a cardiovascular disease or condition. In one embodiment, the chronic wound is a persistent epithelial defect. In one embodiment the first composition is administered first. In another embodiment, the second composition is administered first. In a further embodiment, the method further comprises administration of a third composition, wherein the third wound healing composition comprises a anti-connexin agent, for example, an anti-connexin polynucleotide, peptide or peptidomimetic. In one embodiment the methods of the present invention may be used to treat persistent epithelial defects. Application of the compositions of the present invention may improve healing of the epithelium and basement membrane complex. In one embodiment the third composition is administered first. In one embodiment the third composition is administered first. In one embodiment the pharmaceutical compositions are administered topically.

In a further aspect, the invention provides a method for reducing scar formation in a subject in need thereof, comprising administering to said subject a therapeutically effective amount of a pharmaceutical composition comprising a first anti-connexin agent and a second anti-connexin agent as described herein, for example, one or more anti-connexin polynucleotides and one or more anti-connexin peptides or peptidomimetics. In one embodiment, said method comprises administration of two pharmaceutical compositions, the first composition comprising one or more anti-connexin polynucleotides and the second pharmaceutical composition comprising one or more anti-connexin peptides or peptidomimetics. In one embodiment the first composition is administered first. In another embodiment, the second composition is administered first. In a further embodiment, the method, further comprises administration of a third composition, wherein the third wound healing composition comprises an anti-connexin agent, for example, an anti-connexin polynucleotide, peptide or peptidomimetic. In one embodiment the third composition is administered first. In one embodiment the pharmaceutical compositions are administered topically.

Preferred methods include the sequential or simultaneous administration a first anti-connexin agent and a second anti-connexin agent as described herein, for example, one or more anti-connexin polynucleotides and one or more anti-connexin peptides or peptidomimetics, either or both of which are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

In a further aspect, the invention includes transdermal patches, dressings, pads, wraps, matrices and bandages capable of being adhered or otherwise associated with the skin of a subject, said articles being capable of delivering a therapeutically effective amount of a first anti-connexin agent and a second anti-connexin agent as described herein, for example, one or more anti-connexin polynucleotides and one or more anti-connexin peptides or peptidomimetics to a subject.

In another aspect, the invention includes an article of manufacture comprising a vessel containing a therapeutically effective amount of a first anti-connexin agent and a second anti-connexin agent as described herein, for example, one or more pharmaceutically acceptable anti-connexin polynucleotides and one or more pharmaceutically acceptable anti-connexin peptides or peptidomimetics and instructions for use, including use for the treatment of a subject.

The invention includes an article of manufacture comprising packaging material containing one or more dosage forms containing a first anti-connexin agent and a second anti-connexin agent as described herein, for example, one or more anti-connexin polynucleotides and one or more anti-connexin peptides or peptidomimetics, wherein the packaging material has a label that indicates that the dosage form can be used for a subject having or suspected of having or predisposed to any of the diseases, disorders and/or conditions described or referenced herein, including diseases, disorders and/or conditions characterized in whole or in part by acute, impaired, delayed or chronic wound healing. Such dosage forms include, for example, topical delivery forms and formulations.

The invention includes a formulation comprising a first anti-connexin agent and a second anti-connexin agent as described herein, for example, one or more anti-connexin polynucleotides and one or more anti-connexin peptides or peptidomimetics in amounts effective to promote healing or tissue repair in a subject. The invention includes a formulation comprising a first anti-connexin agent and a second anti-connexin agent as described herein, for example, one or more anti-connexin polynucleotides and one or more anti-connexin peptides or peptidomimetics in amounts effective to promote wound healing in a subject. Such formulations include, for example, topical delivery forms and formulations. Preferred formulations include, for example, a pharmaceutical composition of the invention which is formulated as a foam, spray or gel. In one embodiment, the gel is a polyoxyethylene-polyoxypropylene copolymer-based gel or a carboxymethylcellulose-based gel. In a preferred embodiment, the gel is a pluronic gel.

Preferred formulations include a first anti-connexin agent and a second anti-connexin agent as described herein, for example, one or more anti-connexin polynucleotides and one or more anti-connexin peptides or peptidomimetics, either or both of which are provided in amounts or doses that are less that those used when the agent or agents are administered alone, i.e., when they are not administered in combination, either physically or in the course of treatment of a wound. Such lesser amounts of agents administered or provided in combination are typically from about one-twentieth to about one-tenth the amount or amounts when administered alone, and may be about one-eighth the amount, about one-sixth the amount, about one-fifth the amount, about one-fourth the amount, about one-third the amount, and about one-half the amount when administered alone.

The invention includes methods for the use of therapeutically effective amounts of compositions comprising a first anti-connexin agent and a second anti-connexin agent as described herein, for example, one or more anti-connexin polynucleotides and one or more anti-connexin peptides or peptidomimetics in the manufacture of a medicament. Such medicaments include, for example, topical delivery forms and formulations. Such medicaments include those for the treatment of a subject as disclosed herein. Such medicaments preferably include the reduced amounts of a first anti-connexin agent and a second anti-connexin agent as described herein, for example, one or more anti-connexin polynucleotides and one or more anti-connexin peptides or peptidomimetics, as noted herein.

The invention includes method of preparing a medicament for treating a wound, comprising bringing together and an amount of a first anti-connexin agent and a second anti-connexin agent as described herein, including, for example, a first composition and a second composition wherein said first composition comprises an effective amount of an anti-connexin polynucleotide and said second composition comprises an effective amount of an anti-connexin peptide or peptidomimetic. Other embodiments preparing medicatments that include first and second compositions comprising an anti-connexin polynucleotides, an anti-connexin peptide or peptidomimetic, a gap junction closing compound useful for wound healing, a hemichannel closing compound useful for wound healing, and/or a connexin carboxy-terminal polypeptide useful for wound healing.

The invention includes methods for the use of a therapeutically effective amount of a first anti-connexin agent and a second anti-connexin agent as described herein, for example, one or more anti-connexin polynucleotides and one or more anti-connexin peptides or peptidomimetics in the manufacture of a dosage form. Such dosage forms include, for example, topical delivery forms and formulations. Such dosage forms include those for the treatment of a subject as disclosed herein. Such dosage forms preferably include the reduced amounts of the one or more anti-connexin polynucleotides and one or more anti-connexin peptides or peptidomimetics, as noted herein, or reduced amounts of a gap junction closing compound useful for wound healing, a hemichannel closing compound useful for wound healing, and/or a connexin carboxy-terminal polypeptide useful for wound healing.

In another aspect, the invention provides for the use of a first anti-connexin agent and a second anti-connexin agent as described herein, for example, an anti-connexin polynucleotide (for example, anti-alpha-1 ODN) and an anti-connexin peptide or peptidomimetic, in the manufacture of a pharmaceutical product for the promotion of wound healing in a patient in need thereof.

In certain other aspect, the invention provides: (i) a package comprising an anti-connexin agent together with instructions for use in combination with another anti-connexin agent for the promotion (e.g. decrease in healing time, better wound outcome) of wound healing, (ii) a package comprising one or more anti-connexin polynucleotides together with instructions for use in combination with one or more anti-connexin peptides or peptidomimetics for the promotion of wound healing; and (iii) a package comprising one or more anti-connexin polynucleotides and one or more anti-connexin peptides or peptidomimetics, together with instructions for use in the promotion of wound healing for a chronic wound.

In a one embodiment the pharmaceutical product of the invention is provided in combination with a wound dressing or wound healing promoting matrix. Suitably the wound dressing or matrix is provided including the form of a solid substrate with a first anti-connexin agent and a second anti-connexin agent as described herein, for example, one or more anti-connexin polynucleotides and one or more anti-connexin peptides or peptidomimetics dispersed on or in the solid substrate.

The first anti-connexin agent and second anti-connexin agent as described herein, for example, anti-connexin polypeptides, peptides and peptidomimetics of the invention, may be administered in the same composition or by separate compositions. Preferably, the agents are administered in the reduced amounts as noted herein.

The anti-connexin agents may be administered to the patient simultaneously, sequentially or separately. If administered separately, preferably the a first anti-connexin agent and a second anti-connexin agent as described herein, for example, anti-connexin polynucleotide(s) and anti-connexin peptide(s) or peptidomimetic(s), are administered sequentially. Preferably, the agents are administered sequentially within at least about one-half hour of each other. The agents may also be administered with about one hour of each other, with about one day to about one week of each other, or as otherwise deemed appropriate. Preferably, the anti-connexin agent is administered first. Preferably, a first anti-connexin agent and a second anti-connexin agent as described herein, for example, an anti-connexin peptide or anti-connexin peptidomimetic, e.g., an anti-connexin agent that can block or reduce hemichannel opening, is administered prior to the administration of an anti-connexin polynucleotide that blocks or reduce connexin expression or the formation of hemichannels or gap junctions, e.g., by downregulation of connexin protein expression. Preferably, the anti-connexin agent or agents is/are anti-connexin 43 agent(s).

These and other aspects of the present inventions, which are not limited to or by the information in this Brief Summary, are provided below.

### DETAILED DESCRIPTION

### Definitions

As used herein, a “disorder” is any disorder, disease, or condition that would benefit from an agent that promotes wound healing and/or reduces swelling, inflammation, and/or scar formation. For example, included are wounds resulting from surgery or trauma, and wound-associated abnormalities in connection with neuropathic, ischemic, microvascular pathology, pressure over bony area (tailbone (sacral), hip (trochanteric), buttocks (ischial), or heel of the foot), reperfusion injury, and valve reflux etiology and conditions.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred pet animals are dogs and cats.

As used herein, “preventing” means preventing in whole or in part, or ameliorating or controlling.

As used herein, a “therapeutically effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve the promotion and/or improvement of wound healing, including rates of wound healing and closure of wounds, in whole or in part. Other benefits include decreases in swelling, inflammation and/or scar formation, in whole or in part.

As used herein, the terms “treating” and “treatment” refer to both therapeutic treatment and prophylactic or preventative measures.

As used herein, “anti-connexin agents” are compounds that affect or modulate the activity, expression or formation of a connexin, a connexin hemichannel (connexon), or a gap junction. Anti-connexin agents include, without limitation, antisense compounds (e.g. antisense polynucleotides), RNAi and siRNA compounds, antibodies and binding fragments thereof, and peptides and polypeptides, which include “peptidomimetics,” and peptide analogs. In addition to anti-connexin polynucleotides and anti-connexin peptides or peptidomimetics, other anti-connexin agents include gap junction closing compounds useful for wound healing (e.g., connexin phosphorylation compounds), hemichannel closing compounds useful for wound healing (e.g., connexin phosphorylation compounds), and connexin carboxy-terminal polypeptide useful for wound healing. Preferred anti-connexin agents are anti-connexin 43 agents, anti-connexin 43 gap junction agents, and anti-connexin 43 hemichannel agents. Exemplary anti-connexin agents are discussed in further detail herein.

As used herein, “simultaneously” is used to mean that the one or more agents of the invention are administered concurrently, whereas the term “in combination” is used to mean they are administered, if not simultaneously or in physical combination, then “sequentially” within a timeframe that they both are available to act therapeutically. Thus, administration “sequentially” may permit one agent to be administered within minutes (for example, 1, 2, 3, 4, 5, 10, 15, 20, 25, 30) minutes or a matter of hours, days, weeks or months after the other provided that both the one or more anti-connexin polynucleotides and one or more anti-connexin peptides or peptidomimetics are concurrently present in effective amounts. The time delay between administration or administrations of the components will vary depending on the exact nature of the components, the interaction there between, and their respective half-lives.

The terms “peptidomimetic” and “mimetic” include naturally occurring and synthetic chemical compounds that may have substantially the same structural and functional characteristics of protein regions which they mimic. In the case of connexins, these may mimic, for example, the extracellular loops of opposing connexins involved in connexon-connexon docking and cell-cell channel formation, and/or the extracellular loops of hemichannel connexins.

As used herein, the term “peptide analogs” refer to the compounds with properties analogous to those of the template peptide and can be non-peptide drugs. “Peptidomimetics” (also known as peptide mimetics) which include peptide-based compounds, also include such non-peptide based compounds such as peptide analogs. Peptidomimetics that are structurally similar to therapeutically useful peptides can be used to produce an equivalent or enhanced therapeutic or prophylactic effect. Generally, peptidomimetics are structural or functional mimics (e.g. identical or similar) to a paradigm polypeptide (i.e., a polypeptide that has a biological or pharmacological function or activity), but can also have one or more peptide linkages optionally replaced by a linkage selected from the group consisting of, for example, —CH2NH—, —CH2S—, —CH2-CH2-, —CH═CH— (cis and trans), —COCH2-, —CH(OH)CH2-, and —CH2SO—. The mimetic can be either entirely composed of natural amino acids, synthetic chemical compounds, non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. In the case of connexins, these can mimic, for example, the extracellular loops of opposing connexins involved in connexon-connexon docking and cell-cell channel formation. For example, a mimetic composition can be useful as a gap junction modulating agent if it is capable of down-regulating biological actions or activities of connexons, such as, for example, preventing the docking of connexons to form gap-junction-mediated cell-cell communications, or preventing the opening of connexons to expose the cell cytoplasm to the extracellular millieu. Peptidomimetics encompass those described herein, as well as those as may be known in the art, whether now known or later developed.

In general, the terms “modulator” and “modulation” of connexin activity, as used herein in its various forms, refers to inhibition in whole or in part of the action or activity of a connexin or a connexin hemichannel or connexin gap junction and may function as anti-connexin agents, including as gap junction modulation agents.

As used herein, the term “protein” refers to any polymer of two or more individual amino acids (whether or not naturally occurring) linked via peptide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. A “domain” of a protein is also a fragment, and comprises the amino acid residues of the protein often required to confer activity or function.

The term “wound dressing” refers to a dressing for topical application to a wound and excludes compositions suitable for systemic administration. For example, the one or more anti-connexin agents, including gap junction modulation agents, may be dispersed in or on a solid sheet of wound contacting material such as a woven or nonwoven textile material, or may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example in a gel or ointment. In certain embodiments the one or more anti-connexin agents, including gap junction modulation agents are dispersed in or on a biodegradable sheet material that provides sustained release of the active ingredients into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

As used herein, “matrix” includes for example, matrices such as collagen, acellular matrices, crosslinked biological scaffold molecules, tissue-based matrices (including pig-based wound healing matrices), cultured epidermal autografts, cultured epidermal allografts, tissue-engineered skin, collagen and glycosaminoglycan dermal matrices inoculated with autologous fibroblasts and keratinocytes, Alloderm (a nonliving allogeneic acellular dermal matrix with intact basement membrane complex), living skin equivalents (e.g., Dermagraft (living allogeneic dermal fibroblasts grown on degradable scaffold), TransCyte (an extracellular matrix generated by allogeneic human dermal fibroblasts), Apligraf (a living allogeneic bilayered construct containing keratinocytes, fibroblasts and bovine type I collagen), and OrCel (allogeneic fibroblasts and keratinocytes seeded in opposite sides of bilayered matrix of bovine collagen), animal derived dressings (e.g., Oasis's porcine small intestinal submucosa acellular collagen matrix; and E-Z Derm's acellular xenogeneic collagen matrix), tissue-based bioengineered structural frameworks, biomanufactured bioprostheses, and other implanted or applied structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson et al., Trans Am Soc Artif Intern 1990; 36:340-343) or other biomatrix which have been engineered to reduce the antigenic response to the xenograft material. Other matrix useful in promotion of wound healing may include for example, processed bovine pericardium proteins comprising insoluble collagen and elastin (Courtman et al., J Biomed Mater Res 1994; 28:655-666) and other acellular tissue which may be useful for providing a natural microenvironment for host cell migration to accelerate tissue regeneration (Malone et al., J Vase Surg 1984; 1:181-91). In certain embodiments, the matrix material may be supplemented with one or more anti-connexin polynucleotides and/or the one or more anti-connexin peptides or peptidomimetics for site specific release of such agents.

As used herein, “wound promoting matrix” includes for example, synthetic or naturally occurring matrices such as collagen, acellular matrix, crosslinked biological scaffold molecules, tissue based bioengineered structural framework, biomanufactured bioprostheses, and other implanted structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson G J et al. (1990) Trans Am Soc Artif Intern 36:340-343) or other biomatrix which have been engineered to reduce the antigenic response to the xenograft material. Other matrices useful in promotion of wound healing may include for example, processed bovine pericardium proteins comprising insoluble collagen and elastin (Courtman D W et al. (1994) J Biomed Mater Res 28:655-666) and other acellular tissue which may be useful for providing a natural microenvironment for host cell migration to accelerate tissue regeneration (Malone J M et al. (1984) J Vase Surg 1:181-91). The invention contemplates a synthetic or natural matrix comprising one or more anti-connexin agents.

### Wounds and Wound Classification

As used herein, the term “wound” includes an injury to any tissue, including, for example, acute, delayed or difficult to heal wounds, and chronic wounds. Examples of wounds may include both open and closed wounds. Wounds include, for example, burns, incisions, excisions, lacerations, abrasions, puncture on penetrating wounds, surgical wounds, contusions, hematoma, crushing injuries and ulcers. Also included are wounds that do not heal at expected rates. The term “wound” may also include for example, injuries to the skin and subcutaneous tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

The term “partial thickness wound” refers to wounds that encompass Grades I-III; examples of partial thickness wounds include pressure sores, venous stasis ulcers, and diabetic ulcers. The present invention contemplates treating all wounds of a type that do not heal at expected rates, including, delayed-healing wounds, incompletely healing wounds, and chronic wounds.

By “wound that does not heal at an/the expected rate” is meant an injury to any tissue that does not heal in an expected or typical time frame, including delayed or difficult to heal wounds (including delayed or incompletely healing wounds), and chronic wounds. Examples of wounds that do not heal at the expected rate include ulcers such as diabetic ulcers, diabetic foot ulcers, vascultic ulcers, arterial ulcers, venous ulcers, venous stasis ulcers, burn ulcers, infectious ulcers, trauma-induced ulcers, pressure ulcers, decubitus ulcers, ulcerations associated with pyoderma gangrenosum, and mixed ulcers. Other wounds that do not heal at expected rates include dehiscent wounds.

As used herein, a delayed or difficult to heal wound may include, for example, a wound that is characterized at least in part by 1) a prolonged inflammatory phase, 2) a slow forming extracellular matrix, and/or 3) a decreased rate of epithelialization or closure.

The term “chronic wound” refers to a wound that has not healed. Wounds that do not heal within three months, for example, are considered chronic. Chronic wounds include, for example, pressure ulcers, decubitus ulcers, diabetic ulcers including diabetic foot and leg ulcers, slow or non-healing venous ulcers, venous stasis ulcers, arterial ulcers, vasculitic ulcers, burn ulcers, trauma-induced ulcers, infectious ulcers, mixed ulcers, and pyoderma gangrenosum. The chronic wound may be an arterial ulcer which comprises ulcerations resulting from complete or partial arterial blockage. The chronic wound may be a venous or venous stasis ulcer which comprises ulcerations resulting from a malfunction of the venous valve and the associated vascular disease. In certain embodiments a method of treating a chronic wound is provided where the chronic wound is characterized by one or more of the following AHCPR stages of pressure ulceration: stage 1, stage 2, stage 3, and/or stage 4.

As used herein, chronic wound may also include, for example, a wound that is characterized at least in part by 1) a chronic self-perpetuating state of wound inflammation, 2) a deficient and defective wound extracellular matrix, 3) poorly responding (senescent) wound cells (including fibroblasts), 4) limited extracellular matrix production, and/or 5) failure of re-epithelialization due in part to lack of the necessary extracellular matrix orchestration and lack of scaffold for migration. Chronic wounds may also be characterized by 1) prolonged inflammation and proteolytic activity leading to ulcerative lesions, including for example, diabetic, pressure (decubitous), venous, and arterial ulcers; 2) progressive deposition of matrix in the affected area, 3) longer repair times, 4) less wound contraction, 5) slower re-epithelialization, and 6) increased thickness of granulation tissue.

Exemplary chronic wounds may include “pressure ulcer.” Exemplary pressure ulcers may include all 4 stages of wound classifications based on AHCPR (Agency for Health Care Policy and Research, U.S. Department of Health and Human Services) guidelines, including for example, Stage 1. A stage I pressure ulcer is an observable pressure related alteration of intact skin whose indicators as compared to the adjacent or opposite area on the body may include changes in one or more of the following: skin temperature (warmth or coolness), tissue consistency (firm or boggy feel) and/or sensation (pain, itching). The ulcer appears as a defined area of persistent redness in lightly pigmented skin, whereas in darker skin tones, the ulcer may appear with persistent red, blue, or purple hues. Stage 1 ulceration may include nonblanchable erythema of intact skin and the heralding lesion of skin ulceration. In individuals with darker skin, discoloration of the skin, warmth, edema, induration, or hardness may also be indicators of stage 1 ulceration. Stage 2: stage 2 ulceration may be characterized by partial thickness skin loss involving epidermis, dermis, or both. The ulcer is superficial and presents clinically as an abrasion, blister, or shallow crater. Stage 3: stage 3 ulceration may be characterized by full thickness skin loss involving damage to or necrosis of subcutaneous tissue that may extend down to, but not through, underlying fascia. The ulcer presents clinically as a deep crater with or without undermining of adjacent tissue. Stage 4: stage 4 ulceration may be characterized by full thickness skin loss with extensive destruction, tissue necrosis, or damage to muscle, bone, or supporting structures (e.g., tendon, joint capsule). In certain embodiments a method of treating a chronic wound is provided where the chronic wound is characterized by one or more of the following AHCPR stages of pressure ulceration: stage 1, stage 2, stage 3, and/or stage 4.

Exemplary chronic wounds may include “decubitus ulcers.” Exemplary decubitus ulcers may arise as a result of prolonged and unrelieved pressure over a bony prominence that leads to ischemia. The wound tends to occur in patients who are unable to reposition themselves to off-load weight, such as paralyzed, unconscious, or severely debilitated persons. As defined by the U.S. Department of Health and Human Services, the major preventive measures include identification of high-risk patients; frequent assessment; and prophylactic measures such as scheduled repositioning, appropriate pressure-relief bedding, moisture barriers, and adequate nutritional status. Treatment options may include for example, pressure relief, surgical and enzymatic debridement, moist wound care, and control of the bacterial load. In certain embodiments a method of treating a chronic wound is provided wherein the chronic wound is characterized by decubitus ulcer or ulceration which results from prolonged, unrelieved pressure over a bony prominence that leads to ischemia. In certain embodiments a method of treating a chronic wound is provided wherein the chronic wound is characterized by decubitus ulcer or ulceration which results from prolonged, unrelieved pressure over a bony prominence that leads to ischemia.

Exemplary chronic wounds may include “arterial ulcers.” Chronic arterial ulcers are generally understood to be ulcerations that accompany arteriosclerotic and hypertensive cardiovascular disease. They are painful, sharply marginated, and often found on the lateral lower extremities and toes. Arterial ulcers may include those ulcers characterized by complete or partial arterial blockage which may lead to tissue necrosis and/or ulceration. Signs of arterial ulcer may include, for example, pulselessness of the extremity; painful ulceration; small, punctate ulcers that are usually well circumscribed; cool or cold skin; delayed capillary return time (briefly push on the end of the toe and release, normal color should return to the toe in about 3 seconds or less); atrophic appearing skin (for example, shiny, thin, dry); and loss of digital and pedal hair.

Exemplary chronic wounds may include “venous ulcers.” Exemplary venous ulcers may include the most common type of ulcer affecting the lower extremities and may be characterized by malfunction of the venous valve. The normal vein has valves that prevent the backflow of blood. When these valves become incompetent, the backflow of venous blood causes venous congestion. Hemoglobin from the red blood cells escapes and leaks into the extravascular space, causing the brownish discoloration commonly noted. It has been shown that the transcutaneous oxygen pressure of the skin surrounding a venous ulcer is decreased, suggesting that there are forces obstructing the normal vascularity of the area. Lymphatic drainage and flow also plays a role in these ulcers. The venous ulcer may appear near the medial malleolus and usually occurs in combination with an edematous and indurated lower extremity; it may be shallow, not too painful and may present with a weeping discharge from the affected site. In certain embodiments a method of treating a chronic wound is provided wherein the chronic wound is characterized by arterial ulcers or ulcerations due to complete or partial arterial blockage.

Exemplary chronic wounds may include “venous stasis ulcers.” Stasis ulcers are lesions associated with venous insufficiency are more commonly present over the medial malleolus, usually with pitting edema, varicosities, mottled pigmentation, erythema, and nonpalpable petechiae and purpura. The stasis dermatitis and ulcers are generally pruritic rather than painful. Exemplary venous stasis ulcers may be characterized by chronic passive venous congestion of the lower extremities results in local hypoxia. One possible mechanism of pathogenesis of these wounds includes the impediment of oxygen diffusion into the tissue across thick perivascular fibrin cuffs. Another mechanism is that macromolecules leaking into the perivascular tissue trap growth factors needed for the maintenance of skin integrity. Additionally, the flow of large white blood cells slows due to venous congestion, occluding capillaries, becoming activated, and damaging the vascular endothelium to predispose to ulcer formation. In certain embodiments a method of treating a chronic wound is provided wherein the chronic wound is characterized by venous ulcers or ulcerations due to malfunction of the venous valve and the associated vascular disease. In certain embodiments a method of treating a chronic wound is provided wherein the chronic wound is characterized by venous stasis ulcers or ulcerations due to chronic passive venous congestion of the lower extremities and/or the resulting local hypoxia.

Exemplary chronic wounds may include “diabetic ulcers.” Diabetic patients are prone to ulcerations, including foot ulcerations, due to both neurologic and vascular complications. Peripheral neuropathy can cause altered or complete loss of sensation in the foot and/or leg. Diabetic patients with advanced neuropathy loose all ability for sharp-dull discrimination. Any cuts or trauma to the foot may go completely unnoticed for days or weeks in a patient with neuropathy. It is not uncommon to have a patient with neuropathy notice that the ulcer “just appeared” when, in fact, the ulcer has been present for quite some time. For patients of neuropathy, strict glucose control has been shown to slow the progression of the disease. Charcot foot deformity may also occur as a result of decreased sensation. People with “normal” feeling in their feet have the ability to sense automatically when too much pressure is being placed on an area of the foot. Once identified, our bodies instinctively shift position to relieve this stress. A patient with advanced neuropathy looses this ability to sense the sustained pressure insult, as a result, tissue ischemia and necrosis may occur leading to for example, plantar ulcerations. Additionally, microfractures in the bones of the foot, if unnoticed and untreated, may result in disfigurement, chronic swelling and additional bony prominences. Microvascular disease is one of the significant complications for diabetics which may also lead to ulcerations. In certain embodiments a method of treating a chronic wound is provided wherein the chronic wound is characterized by diabetic foot ulcers and/or ulcerations due to both neurologic and vascular complications of diabetes.

Exemplary chronic wounds can include “traumatic ulcers.” Formation of exemplary traumatic ulcers may occur as a result of traumatic injuries to the body. These injuries include, for example, compromises to the arterial, venous or lymphatic systems; changes to the bony architecture of the skeleton; loss of tissue layers-epidermis, dermis, subcutaneous soft tissue, muscle or bone; damage to body parts or organs and loss of body parts or organs. In certain embodiments, a method of treating a chronic wound is provided wherein the chronic wound is characterized by ulcerations associated with traumatic injuries to the body.

Exemplary chronic wounds can include “burn ulcers” including for example, ulceration that occur as a result of a burn injury, including 1st degree burn (i.e. superficial, reddened area of skin); 2nd degree burn (a blistered injury site which may heal spontaneously after the blister fluid has bee removed); 3rd degree burn (burn through the entire skin and usually require surgical intervention for wound healing); scalding (may occur from scalding hot water, grease or radiator fluid); thermal (may occur from flames, usually deep burns); chemical (may come from acid and alkali, usually deep burns); electrical (either low voltage around a house or high voltage at work); explosion flash (usually superficial injuries); and contact burns (usually deep and may occur from muffler tail pipes, hot irons and stoves). In certain embodiments, a method of treating a chronic wound is provided wherein the chronic wound is characterized by ulcerations associated with burn injuries to the body.

Exemplary chronic wounds can include “vasculitic ulcers.” Vasculitic ulcers also occur on the lower extremities and are painful, sharply marginated lesions, which may have associated palpable purpuras and hemorrhagic bullae. The collagen diseases, septicemias, and a variety of hematological disorders (e.g., thrombocytopenia, dysproteinemia) may be the cause of this severe, acute condition.

Exemplary chronic wounds can include pyoderma gangrenosum. Pyoderma gangrenosum occurs as single or multiple, very tender ulcers of the lower legs. A deep red to purple, undermined border surrounds the purulent central defect. Biopsy typically fails to reveal a vasculitis. In half the patients it is associated with a systemic disease such as ulcerative colitis, regional ileitis, or leukemia. In certain embodiments, a method of treating a chronic wound is provided wherein the chronic wound is characterized by ulcerations associated with pyoderma gangrenosum.

Exemplary chronic wounds can include ocular ulcers, including corneal ulcers or indolent ulcers. Also included are persistent epithelial defects. These may occur in humans and also in sport animals (such as horses) and pet animals (including dogs).

Exemplary chronic wounds can include infectious ulcers. Infectious ulcers follow direct innoculation with a variety of organisms and may be associated with significant regional adenopathy. Mycobacteria infection, anthrax, diphtheria, blastomyosis, sporotrichosis, tularemia, and cat-scratch fever are examples. The genital ulcers of primary syphilis are typically nontender with a clean, firm base. Those of chancroid and granuloma inguinale tend to be ragged, dirty, and more extravagant lesions. In certain embodiments, a method of treating a chronic wound is provided wherein the chronic wound is characterized by ulcerations associated with infection.

As used herein, the term “dehiscent wound” refers to a wound, usually a surgical wound, which has ruptured or split open. In certain embodiments, a method of treating a wound that does not heal at the expected rate is provided wherein the wound is characterized by dehiscence.

In addition to the definition previously provided, the term “wound” may also include for example, injuries to the skin and subcutaneous tissue initiated in different ways (e.g., pressure sores from extended bed rest and wounds induced by trauma) and with varying characteristics.

### Anti-Connexin Agents

Anti-connexin agents of the invention described herein are capable of modulating or affecting the transport of molecules into and out of cells (e.g., blocking or inhibiting or downregulating). Thus, certain anti-connexin agents described herein modulate cellular communication (e.g., cell to cell). Certain anti-connexin agents are gap junction modulation agents. Certain anti-connexin agents modulate or effect transmission of molecules between the cell cytoplasm and the periplasmic or extracellular space. Such anti-connexin agents are generally targeted to connexins and/or connexin hemichannels (connexons). Hemichannels and resulting gap junctions that comprise connexins are independently involved in the release or exchange of small molecules between the cell cytoplasm and an extracellular space or tissue in the case of open hemichannels, and between the cytoplasm of adjoining cell in the case of open gap junctions. Thus, an anti-connexin agents provided herein may directly or indirectly reduce coupling and communication between cells or reduce or block communication (or the transmission of molecules) between a cell and extracellular space or tissue, and the modulation of transport of molecules from a cell into an extracellular space or tissue (or from an extracellular space or tissue into a cell) or between adjoining cells is within the scope of anti-connexin agents and embodiments of the invention. Preferably, the connexin is connexin 43.

Any anti-connexin agent that is capable of eliciting a desired inhibition of the passage (e.g. transport) of molecules through a gap junction or connexin hemichannel may be used in embodiments of the invention. Any anti-connexin agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm). Such anti-connexin agents may modulate the passage of molecules through a gap junction or connexin hemichannel with or without gap junction uncoupling (blocking the transport of molecules through gap junctions). Such compounds include, for example, proteins and polypeptides, polynucleotides, and other organic compounds, and they may, for example block the function or expression of a gap junction or a hemichannel in whole or in part, or downregulate the production of a connexin in whole or in part. Certain gap junction inhibitors are listed in Evans, W. H. and Boitano, S. Biochem. Soc. Trans. 29: 606-612 (2001). Other compounds include connexin phosphorylation compounds that close gap junctions and/or hemichannels, in whole or in part, and connexin carboxy-terminal polypeptides. Preferably, the connexin is connexin 43.

Certain anti-connexin agents provide downregulation of connexin expression (for example, by downregulation of mRNA transcription or translation) or otherwise decrease or inhibit the activity of a connexin protein, a connexin hemichannel or a gap junction. In the case of downregulation, this will have the effect of reducing direct cell-cell communication by gap junctions, or exposure of cell cytoplasm to the extracellular space by hemichannels, at the site at which connexin expression is downregulated. Anti-connexin 43 agents are preferred.

Examples of anti-connexin agents include agents that decrease or inhibit expression or function of connexin mRNA and/or protein or that decrease activity, expression or formation of a connexin, a connexin hemichannel or a gap junction. Anti-connexin agents include anti-connexin polynucleotides, such as antisense polynucleotides and other polynucleotides (such as polynucleotides having siRNA or ribozyme functionalities), as well as antibodies and binding fragments thereof, and peptides and polypeptides, including peptidomimetics and peptide analogs that modulate hemichannel or gap junction activity or function. Anti-connexin 43 agents are preferred.

### Anti-Connexin Polynucleotides

Anti-connexin polynucleotides include connexin antisense polynucleotides as well as polynucleotides which have functionalities which enable them to down-regulate connexin expression. Other suitable anti-connexin polynucleotides include RNAi polynucleotides and siRNA polynucleotides. Anti-connexin 43 polynucleotides are preferred.

Synthesis of antisense polynucleotides and other anti-connexin polynucleotides such as RNAi, siRNA, and ribozyme polynucleotides as well as polynucleotides having modified and mixed backbones is known to those of skill in the art. See e.g. Stein C. A. and Krieg A. M. (eds), Applied Antisense Oligonucleotide Technology, 1998 (Wiley-Liss). Methods of synthesizing antibodies and binding fragments as well as peptides and polypeptides, including peptidomimetics and peptide analogs are known to those of skill in the art. See e.g. Lihu Yang et al., Proc. Natl. Acad. Sci. U.S.A., 1; 95(18): 10836-10841 (Sep. 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA, preferably connexin 43 mRNA. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain aspects, suitable polynucleotides are typically from about 6 to 40 nucleotides in length. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000 or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 43 coding sequence shown in Table 8. The connexin is typically an α or β connexin. Preferably the connexin is an α connexin and is expressed in the tissue to be treated.

Some connexin proteins are however more ubiquitous than others in terms of distribution in tissue. One of the most widespread is connexin 43. Polynucleotides targeted to connexin 43 are particularly suitable for use in the present invention. In other aspects other connexins are targeted.

Anti-connexin polynucleotides include connexin antisense polynucleotides as well as polynucleotides which have functionalities which enable them to down-regulate connexin expression. Other suitable anti-connexin polynucleotides include RNAi polynucleotides and SiRNA polynucleotides.

In one preferred aspect, the antisense polynucleotides are targeted to the mRNA of one connexin protein only. Most preferably, this connexin protein is connexin 43. In another aspect, connexin protein is connexin 26, 30, 31.1, 32, 36, 37, 40, or 45. In other aspects, the connexin protein is connexin 30.3, 31, 40.1, or 46.6.

It is also contemplated that polynucleotides targeted to separate connexin proteins be used in combination (for example 1, 2, 3, 4 or more different connexins may be targeted). For example, polynucleotides targeted to connexin 43, and one or more other members of the connexin family (such as connexin 26, 30, 30.3, 31.1, 32, 36, 37, 40, 40.1, 45, and 46.6) can be used in combination.

Alternatively, the antisense polynucleotides may be part of compositions which may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin, or may target 1, 2, 3 or more different connexins. Specific polynucleotides will generally target sequences in the connexin gene or mRNA which are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30 mer polynucleotide has been found to be particularly suitable.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ GTA ATT GCG GCA AGA AGA ATT GTT TCT GTC 3′ | (connexin 43) | (SEQ.ID.NO: 1) |
| 5′ GTA ATT GCG GCA GGA GGA ATT GTT TCT GTC 3′ | (connexin 43) | (SEQ.ID.NO: 2) |
| 5′ GGC AAG AGA CAC CAA AGA CAC TAC CAG CAT 3′ | (connexin 43) | (SEQ.ID.NO: 3) |
| 5′ TCC TGA GCA ATA CCT AAC GAA CAA ATA 3′ | (connexin 26) | (SEQ.ID.NO: 4) |
| 5′ CAT CTC CTT GGT GCT CAA CC 3′ | (connexin 37) | (SEQ.ID.NO: 5) |
| 5′ CTG AAG TCG ACT TGG CTT GG 3′ | (connexin 37) | (SEQ.ID.NO: 6) |
| 5′ CTC AGA TAG TGG CCA GAA TGC 3′ | (connexin 30) | (SEQ.ID.NO: 7) |
| 5′ TTG TCC AGG TGA CTC CAA GG 3′ | (connexin 30) | (SEQ.ID.NO: 8) |
| 5′ CGT CCG AGC CCA GAA AGA TGA GGT C 3′ | (connexin 31.1) | (SEQ.ID.NO: 9) |
| 5′ AGA GGC GCA CGT GAG ACA C 3′ | (connexin 31.1) | (SEQ.ID.NO: 10) |
| 5′ TGA AGA CAA TGA AGA TGT T 3′ | (connexin 31.1) | (SEQ.ID.NO: 11) |
| 5′ TTT CTT TTC TAT GTG CTG TTG GTG A 3′ | (connexin 32) | (SEQ.ID.NO: 12) |

Suitable polynucleotides for the preparation of the combined polynucleotide compositions described herein include for example, polynucleotides to Connexin Cx43 and polynucleotides for connexins 26, 30, 31.1, 32 and 37 as described in Table 1 above.

Although the precise sequence of the antisense polynucleotide used in the invention will depend upon the target connexin protein, for connexin 43, antisense polynucleotides having the following sequences have been found to be particularly suitable:

(SEQ.ID.NO: 1)

GTA ATT GCG GCA AGA AGA ATT GTT TCT GTC;

(SEQ.ID.NO: 2)

GTA ATT GCG GCA GGA GGA ATT GTT TCT GTC;
and

(SEQ.ID.NO: 3)

GGC AAG AGA CAC CAA AGA CAC TAC CAG CAT.

For example, suitable antisense polynucleotides for connexins 26, 31.1 and 32 have the following sequences:

(SEQ.ID.NO: 4)

5′ TCC TGA GCA ATA CCT AAC GAA CAA ATA
(connexin 26);

(SEQ.ID.NO: 9)

5′ CGT CCG AGC CCA GAA AGA TGA GGT C
(connexin 31.1);
and

(SEQ.ID.NO: 12)

5′ TTT CTT TTC TAT GTG CTG TTG GTG A
(connexin 32).

Other connexin antisense polynucleotide sequences useful according to the methods of the present invention include:

(SEQ.ID.NO: 5)

5′ CAT CTC CTT GGT GCT CAA CC 3′ (connexin 37);

(SEQ.ID.NO: 6)

5′ CTG AAG TCG ACT TGG CTT GG 3′ (connexin 37);

(SEQ.ID.NO: 7)

5′ CTC AGA TAG TGG CCA GAA TGC 3′ (connexin 30)

(SEQ.ID.NO: 8)

5′ TTG TCC AGG TGA CTC CAA GG 3′ (connexin 30);

(SEQ.ID.NO: 10)

5′ AGA GGC GCA CGT GAG ACA C 3′ (connexin 31.1);
and

(SEQ.ID.NO: 11)

5′ TGA AGA CAA TGA AGA TGT T 3′ (connexin 31.1).

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

Polynucleotide Homologues

Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 1091540919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Anti-Connexin Agents

Binding proteins, including peptides, peptidomimetics, antibodies, antibody fragments, and the like, are also suitable modulators of gap junctions and hemichannels.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, hemichannel, or associated molecules).

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10⁻¹⁰ M⁻¹, about 10¹¹ M⁻¹, and about 10¹² M⁻¹. Affinities of binding proteins according to the present invention can be readily determined using conventional techniques, for example those described by Scatchard et al., 1949 Ann. N.Y. Acad. Sci. 51: 660.

By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. J Cell Biol 107: 1817-1824 (1988); Meyer R. A., J Cell Biol 119: 179-189 (1992).

The extracellular domains of a hemichannel contributed by two adjacent cells “dock” with each other to form complete gap junction channels. Reagents that interfere with the interactions of these extracellular domains can impair cell-to-cell communication. Peptide inhibitors of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). Short peptides corresponding to sequences within the extracellular loops of connexins were said to inhibit intercellular communication. Boitano S. and Evans W. Am J Physiol Lung Cell Mol Physicol 279: L623-L630 (2000). The use of peptides as inhibitors of cell-cell channel formation produced by connexin (Cx) 32 expressed in paired Xenopus oocytes has also been reported. Dahl G, et al., Biophys J 67: 1816-1822 (1994). Berthoud, V. M. and Seul, K. H., summarized some of these results. Am J, Physiol. Lung Cell Mol. Physiol. 279: L619-L622 (2000).

Anti-connexin agents include peptides comprising an amino acid sequence corresponding to a transmembrane region (e.g. 1^{st} to 4^{th}) of a connexin (e.g. connexin 45, 43, 26, 30, 31.1, and 37). Anti-connexin agents may comprise a peptide comprising an amino acid sequence corresponding to a portion of a transmembrane region of a connexin 45. Anti-connexin agents include a peptide having an amino acid sequence that comprises about 5 to 20 contiguous amino acids of SEQ. ID. NO.:13, a peptide having an amino acid sequence that comprises about 8 to 15 contiguous amino acids of SEQ. ID. NO.:13, or a peptide having an amino acid sequence that comprises about 11 to 13 contiguous amino acids of SEQ. ID. NO.:13. Other embodiments are directed to an anti-connexin agent that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO.:13. In certain anti-connexin agents provided herein, the extracellular domains of connexin 45 corresponding to the amino acids at positions 46-75 and 199-228 of SEQ ID NO: 13 may be used to develop the particular peptide sequences. Certain peptides described herein have an amino acid sequence corresponding to the regions at positions 46-75 and 199-228 of SEQ. ID. NO: 13. The peptides need not have an amino acid sequence identical to those portions of SEQ. ID. NO: 13, and conservative amino acid changes may be made such that the peptides retain binding activity or functional activity. Alternatively, the peptide may target regions of the connexin protein other than the extracellular domains (e.g. the portions of SEQ. ID. NO:13 not corresponding to positions 46-75 and 199-228).

Also, suitable anti-connexin agents comprise a peptide comprising an amino acid sequence corresponding to a portion of a transmembrane region of a connexin 43. Anti-connexin agents include peptides having an amino acid sequence that comprises about 5 to 20 contiguous amino acids of SEQ. ID. NO:14, peptides having an amino acid sequence that comprises about 8 to 15 contiguous amino acids of SEQ. ID. NO:14, or peptides having an amino acid sequence that comprises about 11 to 13 contiguous amino acids of SEQ. ID. NO:14. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:14. Other anti-connexin agents comprise the extracellular domains of connexin 43 corresponding to the amino acids at positions 37-76 and 178-208 of SEQ. ID. NO: 14. Anti-connexin agents include peptides described herein which have an amino acid sequence corresponding to the regions at positions 37-76 and 178-208 of SEQ. ID. NO: 14. The peptides need not have an amino acid sequence identical to those portions of SEQ. ID. NO: 14, and conservative amino acid changes may be made such that the peptides retain binding activity or functional activity. Alternatively, peptides may target regions of the connexin protein other than the extracellular domains (e.g. the portions of SEQ. ID. NO.:14 not corresponding to positions 37-76 and 178-208).

```
Connexin 45
(SEQ ID NO. 13)
Met Ser Trp Ser Phe Leu Thr Arg Leu Leu Glu Glu Ile His Asn His
1 5 10 15
Ser Thr Phe Val Gly Lys Ile Trp Leu Thr Val Leu Ile Val Phe Arg
                        20 25 30
Ile Val Leu Thr Ala Val Gly Gly Glu Ser Ile Tyr Tyr Asp Glu Gln
                35 40 45
Ser Lys Phe Val Cys Asn Thr Glu Gln Pro Gly Cys Glu Asn Val Cys
        50 55 60
Tyr Asp Ala Phe Ala Pro Leu Ser His Val Arg Phe Trp Val Phe Gln
65 70 75 80
Ile Ile Leu Val Ala Thr Pro Ser Val Met Tyr Leu Gly Tyr Ala Ile
                                85 90 95
His Lys Ile Ala Lys Met Glu His Gly Glu Ala Asp Lys Lys Ala Ala
                        100 105 110
Arg Ser Lys Pro Tyr Ala Met Arg Trp Lys Gln His Arg Ala Leu Glu
                115 120 125
Glu Thr Glu Glu Asp Asn Glu Glu Asp Pro Met Met Tyr Pro Glu Met
        130 135 140
Glu Leu Glu Ser Asp Lys Glu Asn Lys Glu Gln Ser Gln Pro Lys Pro
145 150 155 160
Lys His Asp Gly Arg Arg Arg Ile Arg Glu Asp Gly Leu Met Lys Ile
                                165 170 175
Tyr Val Leu Gln Leu Leu Ala Arg Thr Val Phe Glu Val Gly Phe Leu
                        180 185 190
Ile Gly Gln Tyr Phe Leu Tyr Gly Phe Gln Val His Pro Phe Tyr Val
                195 200 205
Cys Ser Arg Leu Pro Cys Pro His Lys Ile Asp Cys Phe Ile Ser Arg
        210 215 220
Pro Thr Glu Lys Thr Ile Phe Leu Leu Ile Met Tyr Gly Val Thr Gly
225 230 235 240
Leu Cys Leu Leu Leu Asn Ile Trp Glu Met Leu His Leu Gly Phe Gly
                                245 250 255
Thr Ile Arg Asp Ser Leu Asn Ser Lys Arg Arg Glu Leu Glu Asp Pro
                        260 265 270
Gly Ala Tyr Asn Tyr Pro Phe Thr Trp Asn Thr Pro Ser Ala Pro Pro
                275 280 285
Gly Tyr Asn Ile Ala Val Lys Pro Asp Gln Ile Gln Tyr Thr Glu Leu
        290 295 300
Ser Asn Ala Lys Ile Ala Tyr Lys Gln Asn Lys Ala Asn Thr Ala Gln
305 310 315 320
Glu Gln Gln Tyr Gly Ser His Glu Glu Asn Leu Pro Ala Asp Leu Glu
                                325 330 335
Ala Leu Gln Arg Glu Ile Arg Met Ala Gln Glu Arg Leu Asp Leu Ala
                        340 345 350
Val Gln Ala Tyr Ser His Gln Asn Asn Pro His Gly Pro Arg Glu Lys
                355 360 365
Lys Ala Lys Val Gly Ser Lys Ala Gly Ser Asn Lys Ser Thr Ala Ser
        370 375 380
Ser Lys Ser Gly Asp Gly Lys Asn Ser Val Trp Ile
385 390 395
Connexin 43
(SEQ ID NO. 14)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
                        20 25 30
Arg Ile Leu Leu Leu Gly Thr Ala Val Glu Ser Ala Trp Gly Asp Glu
                35 40 45
Gln Ser Ala Phe Arg Cys Asn Thr Gln Gln Pro Gly Cys Glu Asn Val
        50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                                85 90 95
Phe Tyr Val Met Arg Lys Glu Glu Lys Leu Asn Lys Lys Glu Glu Glu
                        100 105 110
Leu Lys Val Ala Gln Thr Asp Gly Val Asn Val Asp Met His Leu Lys
                115 120 125
Gln Ile Glu Ile Lys Lys Phe Lys Tyr Gly Ile Glu Glu His Gly Lys
        130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                                165 170 175
Tyr Gly Phe Ser Leu Ser Ala Val Tyr Thr Cys Lys Arg Asp Pro Cys
                        180 185 190
Pro His Gln Val Asp Cys Phe Leu Ser Arg Pro Thr Glu Lys Thr Ile
                195 200 205
Phe Ile Ile Phe Met Leu Val Val Ser Leu Val Ser Leu Ala Leu Asn
        210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                                245 250 255
Ala Lys Asp Cys Gly Ser Gln Lys Tyr Ala Tyr Phe Asn Gly Cys Ser
                        260 265 270
Ser Pro Thr Ala Pro Leu Ser Pro Met Ser Pro Pro Gly Tyr Lys Leu
                275 280 285
Val Thr Gly Asp Arg Asn Asn Ser Ser Cys Arg Asn Tyr Asn Lys Gln
        290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                                325 330 335
Phe Pro Asp Asp Asn Gln Asn Ser Lys Lys Leu Ala Ala Gly His Glu
                        340 345 350
Leu Gln Pro Leu Ala Ile Val Asp Gln Arg Pro Ser Ser Arg Ala Ser
                355 360 365
Ser Arg Ala Ser Ser Arg Pro Arg Pro Asp Asp Leu Glu Ile
        370 375 380
```

The anti-connexin peptides may comprise sequences corresponding to a portion of the connexin extracellular domains with conservative amino acid substitutions such that peptides are functionally active anti-connexin agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Exemplary peptides targeted to connexin 43 are shown below in Table 2. Ml, 2, 3 and 4 refer to the 1^{st} to 4^{th} transmembrane regions of the connexin 43 protein respectively. E1 and E2 refer to the first and second extracellular loops respectively.

TABLE 2
Peptidic Inhibitors of Intercellular
Communication (cx43)

| | | |
|---|---|---|
| FEVAFLLIQWI | M3 & E2 | (SEQ.ID.NO: 15) |
| LLIQWYTGFSL | E2 | (SEQ.ID.NO: 16) |
| SLSAVYTCKRDPCPHQ | E2 | (SEQ.ID.NO: 17) |
| VDCFLSRPTEKT | E2 | (SEQ.ID.NO: 18) |
| SRPTEKTIFII | E2 & M4 | (SEQ.ID.NO: 19) |
| LGTAVESAWGDEQ | M1 & El | (SEQ.ID.NO: 20) |
| QSAFRCNTQQPG | E1 | (SEQ.ID.NO: 21) |
| QQPGCENVCYDK | E1 | (SEQ.ID.NO: 22) |
| VCYDKSFPISHVR | El | (SEQ.ID.NO: 23) |

Table 3 provides additional exemplary connexin peptides used in inhibiting hemichannel or gap junction function. In other embodiments, conservative amino acid changes are made to the peptides or fragments thereof.

TABLE 3
Additional Peptidic Inhibitors of Intercellular Communication (cx32, cx43)

| | | | AA's and |
|---|---|---|---|
| Connexin | Location | | Sequence |
| Cx32 | El 39-77 | AAESVWGDEIKSSFICNTLQPGCNSVCYDHFFPIS | (SEQ.ID.NO: 24) |
| | | HVR | |
| Cx32 | El 41-52 | ESVWGDEKSSFI | (SEQ.ID.NO: 25) |
| Cx32 | El 52-63 | ICNTLQPGCNSV | (SEQ.ID.NO: 26) |
| Cx32 | El 62-73 | SVCYDHFFPISH | (SEQ.ID.NO: 27) |
| Cx32 | E2 64-188 | RLVKCEAFPCPNTVDCFVSRPTEKT | (SEQ.ID.NO: 28) |
| Cx32 | E2 166-177 | VKCEAFPCPNTV | (SEQ.ID.NO: 29) |
| Cx32 | E2 177-188 | VDCFVSRPTEKT | (SEQ.ID.NO: 30) |
| Cx32 | El 63-75 | VCYDHFFPISHVR | (SEQ.ID.NO: 31) |
| Cx32 | El 45-59 | VWGDEKSSFICNTLQPGY | (SEQ.ID.NO: 32) |
| Cx32 | EI 46-59 | DEKSSFICNTLQPGY | (SEQ.ID.NO: 33) |
| Cx32 | E2 182-192 | SRPTEKTVFTV | (SEQ.ID.NO: 34) |
| Cx32/Cx43 | E2 182-188/ | SRPTEKT | (SEQ.ID.NO: 35) |
| | 201-207 | | |
| Cx32 | E1 52-63 | ICNTLQPGCNSV | (SEQ.ID.NO: 36) |
| Cx40 | E2 177-192 | FLDTLHVCRRSPCPHP | (SEQ.ID.NO: 37) |
| Cx43 | E2 188-205 | KRDPCHQVDCFLSRPTEK | (SEQ.ID.NO: 38) |

Table 4 provides the extracellular loops for connexin family members which are used to develop peptide inhibitors for use as described herein. The peptides and provided in Table 4, and fragments thereof, are used as peptide inhibitors in certain non-limiting embodiments. In other non-limiting embodiments, peptides comprising from about 8 to about 15, or from about 11 to about 13 amino contiguous amino acids of the peptides in this Table 4 are peptide inhibitors. Conservative amino acid changes may be made to the peptides or fragments thereof.

TABLE 4
Extracellular loops for various connexin family members

| | | |
|---|---|---|
| E1 | | |
| huCx26 | KEVWGDEQADFVCNTLQPGCKNVCYDHYFPISHIR | (SEQ.ID.NO: 39) |
| huCx30 | QEVWGDEQEDEVCNTLQPGCKNVCYDHFFPVSHIR | (SEQ.ID.NO: 40) |
| huCx30.3 | EEVWDDEQKDFVCNTKQPGCPNVCYDEFFPVSHVR | (SEQ.ID.NO: 41) |
| huCx31 | ERVWGDEQKDFDCNTKQPGCTNVCYDNYFPISNIR | (SEQ.ID.NO: 42) |
| huCx31.1 | ERVWSDDHKDFDCNTRQPGCSNVCFDEFFPVSHVR | (SEQ.ID.NO: 43) |
| huCx32 | ESVWGDEKSSFICNTLQPGCNSVCYDQFFPISHVR | (SEQ.ID.NO: 44) |
| huCx36 | ESVWGDEQSDFECNTAQPGCTNVCYDQAFPISHIR | (SEQ.ID.NO: 45) |
| huCx37 | ESVWGDEQSDFECNTAQPGCTNVCYDQAFPISHIR | (SEQ.ID.NO: 46) |
| huCx40.1 | RPVYQDEQERFVCNTLQPGCANVCYDVFS PVSHLR | (SEQ.ID.NO: 47) |
| huCx43 | ESAWGDEQSAFRCNTQQPGCENVCYDKSFPISHVR | (SEQ.ID.NO: 48) |
| huCx46 | EDVWGDEQSDFTCNTQQPGCBNVCYBRAFPISHIR | (SEQ.ID.NO: 49) |
| huCx46.6 | EAIYSDEQAKFTCNTRQPGCDNVCYDAFAPLSHVR | (SEQ.ID.NO: 50) |
| huCx40 | ESSWGDEQADFRCDTIQPGCQNVCTDQAFPISHIR | (SEQ.ID.NO: 51) |
| huCx45 | GESIYYDEQSKINCNTEQPGCENVCYDAFAPLSHVR | (SEQ.ID.NO: 52) |
| E2 | | |
| huCx26 | MYVFYVMYDGFSMQRLVKCNAWPCPNTVDCFVSRPTEKT | (SEQ.ID.NO: 53) |
| huCx30 | MYVFYFLYNGYHLPWVLKCGIDPCPNLVDCFISRPTEKT | (SEQ.ID.NO: 54) |
| huCx30.3 | LYIFHRLYKDYDMPRVVACSVEPCPHTVDCYISRPTEKK | (SEQ.ID.NO: 55) |
| huCx31 | LYLLHTLWHGFNMPRLVQCANVAPCPNIVDCYIARPTEKK | (SEQ.ID.NO: 56) |
| huCx31.1 | LYVFHSFYPKYILPPVVKCHADPCPNIVDCFISKPSEKN | (SEQ.ID.NO: 57) |
| huCx32 | MYVFYLLYPGYAMVRLVKCDVYPCPNTVDCFVSRPTEKT | SEQ.ID.NO: 58) |
| huCx36 | LYGWTMEPVFVCQRAPCPYLVDCFVSRPTEKT | (SEQ.ID.NO: 59) |
| huCx37 | LYGWTMEPVFVCQRAPCPYLVDCFVSRPTEKT | (SEQ.ID.NO: 60) |
| huCx40.1 | GALHYFLFGFLAPKKFPCTRPPCTGVVDCYVSRPTSKS | (SEQ.ID.NO: 61) |
| huCx43 | LLIQWYIYGFSLSAVYTCKRDPCPHQVDCFLSRPTEKT | (SEQ.ID.NO: 62) |
| huCx46 | IAGQYFLYGFELKPLYRCDRWPCPNTVDCFISRPTEKT | (SEQ.ID.NO: 63) |
| huCx46.6 | LVGQYLLYGFEVRPFFPCSRQPCPHVVDCFVSRPTEKT | (SEQ.ID.NO: 64) |
| huCx40 | IVGQYFIYG1FLTTLHVCRRSPCPHPVNCYVSRPTEKN | (SEQ.ID.NO: 65) |
| huCx45 | LIGQYFLYGFQVHPFYVCSRLPCHPKIDCFISRPTEKT | (SEQ.ID.NO: 66) |

Table 5 provides the extracellular domain for connexin family members which may be used to develop peptide anti-connexin agents. The peptides and provided in Table 5, and fragments thereof, may also be used as peptide anti-connexin agents. Such peptides may comprise from about 8 to about 15, or from about 11 to about 13 amino contiguous amino acids of the peptide sequence in this Table 5. Conservative amino acid changes may be made to the peptides or fragments thereof.

TABLE 5
Extracellular domains

| | | |
|---|---|---|
| Peptide | VDCFLSRPTEKT | (SEQ.ID.NO: 18) |
| Peptide | SRPTEKTIFII | (SEQ.ID.NO: 19) |
| huCx43 | LLIQWYIYGFSLSAVYTCKRDPCPHQVDCFLSRPTEKTIFII | (SEQ.ID.NO: 67) |
| huCx26 | MYVFYVMYDGFSMORLVKCNAWPCPNTVDCFVSRPTEKTVFTV | (SEQ.ID.NO: 68) |
| huCx30 | YVFYFLYNGYHLPWVLKCGIDPCPNLVDCFISRPTEKTVFTI | (SEQ.ID.NO: 69) |
| huCx30.3 | LYIFHRLYKDYDMPRVVACSVEPCPHTVDCYISRPTEKKVFTY | (SEQ.ID.NO: 70) |
| huCx31 | LYLLHTLWHGFNMPRLVQCANVAPCPNIVDCYIARPTEKKTY | (SEQ.ID.NO: 71) |
| huCx31.1 | LYVFHSFYPKYILPPVVKCHADPCPNIVDCFISKPSEKNIFTL | (SEQ.ID.NO: 72) |
| huCx32 | MYVFYLLYPGYAMVRLVKCDVYPCPNTVDCFVSRPTEKTVFTV | (SEQ.ID.NO: 73) |
| huCx36 | LYGWTMEPVFVCQRAPCPYLVDCFVSRPTEKTIFII | (SEQ.ID.NO: 74) |
| huCx37 | LYGWTMEPVFVCQRAPCPYLVDCFVSRPTEKTIFII | (SEQ.ID.NO: 75) |
| huCx40.1 | GALHYFLFGFLAPKKFPCTRPPCTGVVDCYVSRPTEKSLLML | (SEQ.ID.NO: 76) |
| huCx46 | IAGQYFLYGFELKPLYRCDRWPCPNTVDCFISRPTEKTIFII | (SEQ.ID.NO: 77) |
| huCx46.6 | LVGQYLLYGFEVRPFFPCSRQPCPHVVDCFVSRPTEKTVFLL | (SEQ.ID.NO: 78) |
| huCx40 | IVGQYFIYGIFLTTLHVCRRSPCPHPVNCYSRPTEKNVFIV | (SEQ.ID.NO: 79) |
| huCx45 | LIGQYFLYGFQVHPFYVCSRITCHPKIDCFISRPTEKTIFLL | (SEQ.ID.NO: 80) |

Table 6 provides peptides inhibitors of connexin 40 shown with reference to the extracellular loops (E1 and E2) of connexin 40. The bold amino acids are directed to the transmembrane regions of connexin 40.

TABLE 6
Cx40 peptide inhibitors
E2

| | |
|---|---|
| LGTAAESSWGDEQADFRCDTIQPGCQNVCTWAFPISHIRFWVLQ | (SEQ.ID.NO: 81) |
| LGTAAESSWGDEQA | (SEQ.ID.NO: 82) |
| DEQADFRCDTIQP | (SEQ.ID.NO: 83) |
| TIQPGCQNVCTDQ | (SEQ.ID.NO: 84) |
| VCTDQAFPISHIR | (SEQ.ID.NO: 85) |
| AFPISHIRFWVLQ | (SEQ.ID.NO: 86) |
| E2 | |
| MEVGFIVGQYFIYGIFLTTLHVCRRSPCPHPVNCYVSRPTEKNVFIV | (SEQ.ID.NO: 87) |
| MEVGFIVGQYF | (SEQ.ID.NO: 88) |
| IVGQYFIYGIFL | (SEQ.ID.NO: 89) |
| GIFLTTLHVCRRSP | (SEQ.ID.NO: 90) |
| RRSPCPHPVNCY | (SEQ.ID.NO: 91) |
| VNCYVSRPTEKN | (SEQ.ID.NO: 92) |
| SRPTEKNVFIV | (SEQ.ID.NO: 93) |

Table 7 provides peptides inhibitors of connexin 45 shown with reference to the extracellular loops (E1 and E2) of connexin 45. The bold amino acids are directed to the transmembrane regions of connexin 45

TABLE 7
Cx45 peptide inhibitors
El

| | |
|---|---|
| LTAVGGESIYYDEQSKFVCNTEQPGCENVCYDAFAPLSHVRFWVFQ | (SEQ.ID.NO: 94) |
| LTAVGGESIYYDEQS | (SEQ.ID.NO: 95) |
| DEQSKFVCNTEQP | (SEQ.ID.NO: 96) |
| TEQPGCENVCYDA | (SEQ.ID.NO: 97) |
| VCYDAFAPLSHVR | (SEQ.ID.NO: 98) |
| APLSHVRFWVFQ | (SEQ.ID.NO: 99) |

E2

| | |
|---|---|
| FEVGFLIGQYFLYGFQVHPFYVCSRLPCHPKIDCFISRPTEKTIFLL | (SEQ.ID.NO: 100) |
| FEVGFLIGQYF | (SEQ.ID.NO: 101) |
| LIGQYFLYGFQV | (SEQ.ID.NO: 102) |
| GFQVHPFYVCSRLP | (SEQ.ID.NO: 103) |
| SRLPCHPKIDCF | (SEQ.ID.NO: 104) |
| IDCFISRPTEKT | (SEQ.ID.NO: 105) |
| SRPTEKTIFLL | (SEQ.ID.NO: 106) |

In certain embodiments, it is preferred that certain peptide inhibitors block hemichannels without disrupting existing gap junctions. While not wishing to be bound to any particular theory or mechanism, it is also believed that certain peptidomimetics (e.g. VCYDKSFPISHVR, (SEQ. ID. NO: 23) block hemichannels without causing uncoupling of gap junctions (See Leybeart et al., Cell Commun. Adhes. 10: 251-257 (2003)), or do so in lower dose amounts. The peptide SRPTEKTIFII (SEQ. ID. NO: 19) may also be used, for example to block hemichannels without uncoupling of gap junctions. The peptide SRGGEKNVFIV (SEQ. ID. NO: 107) may be used that as a control sequence (DeVriese et al., Kidney Internat. 61: 177-185 (2002)). Examples of peptide inhibitors for connexin 45 YVCSRLPCHP (SEQ. ID. NO:108), QVHPFYVCSRL (SEQ. ID. NO.:109), FEVGFLIGQYFLY (SEQ. ID. NO.:110), GQYFLYGFQVHP (SEQ. ID. NO.:111), GFQVHPFYVCSR (SEQ. ID. NO.:112), AVGGESIYYDEQ (SEQ. ID. NO. 113), YDEQSKFVCNTE (SEQ. ID. NO.:114), NTEQPGCENVCY (SEQ. ID. NO.:115), CYDAFAPLSHVR (SEQ. ID. NO.:116), FAPLSHVRFWVF (SEQ. ID. NO.:117) and LIGQY (SEQ. ID. NO.:118), QVHPF (SEQ. ID. NO.:119), YVCSR (SEQ. ID. NO: 120), SRLPC (SEQ. ID. NO.:121), LPCHP (SEQ. ID. NO.:122) and GESIY (SEQ. ID. NO.:123), YDEQSK (SEQ. ID. NO.:124), SKFVCN (SEQ. ID. NO.:125), TEQPGCEN (SEQ. ID. NO.:126), VCYDAFAP (SEQ. ID. NO.:127), LSHVRFWVFQ (SEQ. ID. NO.:128) The peptides may only be 3 amino acids in length, including SRL, PCH, LCP, CHP, IYY, SKF, QPC, VCY, APL, HVR, or longer, for example: LIQYFLYGFQVHPF (SEQ. ID. NO.:129), VHPFYCSRLPCHP (SEQ. ID. NO: 130), VGGESIYYDEQSKFVCNTEQPG (SEQ. ID. NO.:131), TEQPGCENVCYDAFAPLSHVRF (SEQ. ID. NO.:132), AFAPLSHVRFWVFQ (SEQ. ID. NO: 133).

TABLE 8
Table 8A
Human Connexin 43 from GenBank Accession No. M65188

(SEQ.ID.NO: 134)

| | |
|---|---|
| 1 | ggcttttagc gtgaggaaag taccaaacag cagcggagtt ttaaacttta aatagacagg |
| 61 | tctgagtgcc tgaacttgcc ttttcatttt acttcatcct ccaaggagtt caatcacttg |
| 121 | gcgtgacttc actactttta agcaaaagag tggtgcccag gcaacatggg tgactggagc |
| 181 | gccttaggca aactccttga caaggttcaa gcctactcaa ctgctggagg gaaggtgtgg |
| 241 | ctgtcagtac ttttcatttt ccgaatcctg ctgctgggga cagcggttga gtcagcctgg |
| 301 | ggagatgagc agtctgcctt tcgttgtaac actcagcaac ctggttgtga aaatgtctgc |
| 361 | tatgacaagt ctttcccaat ctctcatgtg cgcttctggg tcctgcagat catatttgtg |
| 421 | tctgtaccca cactcttgta cctggctcat gtgttctatg tgatgcgaaa ggaagagaaa |
| 481 | ctgaacaaga aagaggaaga actcaaggtt gcccaaactg atggtgtcaa tgtggacatg |
| 541 | cacttgaagc agattgagat aaagaagttc aagtacggta ttgaagagca tggtaaggtg |
| 601 | aaaatgcgag gggggttgct gcgaacctac atcatcagta tcctcttcaa gtctatcttt |
| 661 | gaggtggcct tcttgctgat ccagtggtac atctatggat tcagcttgag tgctgtttac |
| 721 | acttgcaaaa gagatccctg cccacatcag gtggactgtt tcctctctcg ccccacggag |
| 781 | aaaaccatct tcatcatatt catgctggtg gtgtccttgg tgtccctggc cttgaatatc |
| 841 | attgaactct tctatgtttt cttcaagggc gttaaggatc gggttaaggg aaagagcgac |
| 901 | ccttaccatg cgaccagtgg tgcgctgagc cctgccaaag actgtgggtc tcaaaaatat |
| 961 | gcttatttca atggctgctc ctcaccaacc gctcccctct cgcctatgtc tcctcctggg |
| 1021 | tacaagctgg ttactggcga cagaaacaat tcttcttgcc gcaattacaa caagcaagca |
| 1081 | agtgagcaaa actgggctaa ttacagtgca gaacaaaatc gaatggggca ggcgggaagc |
| 1141 | accatctcta actcccatgc acagcctttt gatttccccg atgataacca gaattctaaa |
| 1201 | aaactagctg ctggacatga attacagcca ctagccattg tggaccagcg accttcaagc |
| 1261 | agagccagca gtcgtgccag cagcagacct cggcctgatg acctggagat ctag |

Table 8B
Human Connexin 43

(SEQ.ID.NO: 135)

| | |
|---|---|
| 1 | atgggtgactggagcgcctt aggcaaactc cttgacaagg ttcaagccta ctcaactgct |
| 61 | ggagggaaggtgtggctgtc agtacttttc attttccgaatcctgctgct ggggacagcg |
| 121 | gttgagtcagcctggggaga tgagcagtct gcctttcgtt gtaacactca gcaacctggt |
| 181 | tgtgaaaatg tctgctatga caagtctttcccaatctctc atgtgcgctt ctgggtcctg |
| 241 | cagatcatat ttgtgtctgt acccacactcttgtacctgg ctcatgtgttctatgtgatg |
| 301 | cgaaaggaag agaaactgaa caagaaagag gaagaactca aggttgccca aactgatggt |
| 361 | gtcaatgtgg acatgcactt gaagcagatt gagataaagaagttcaagta cggtattgaa |
| 421 | gagcatggta aggtgaaaat gcgagggggg ttgctgcgaa cctacatcat cagtatcctc |
| 481 | ttcaagtcta tctttgaggt ggccttcttg ctgatccagt ggtacatcta tggattcagc |
| 541 | ttgagtgctg tttacacttg caaaagagat ccctgcccac atcaggtgga ctgtttcctc |
| 601 | tctcgcccca cggagaaaac catcttcatc atcttcatgc tggtggtgtc cttggtgtcc |
| 661 | ctggccttga atatcattga actcttctat gttttcttca agggcgttaa ggatcgggtt |
| 721 | aagggaaaga gcgaccctta ccatgcgacc agtggtgcgc tgagccctgc caaagactgt |
| 781 | gggtctcaaa aatatgctta tttcaatggc tgctcctcac caaccgctcc cctctcgcct |
| 841 | atgtctcctc ctgggtacaa gctggttact ggcgacagaa acaattcttc ttgccgcaat |
| 901 | tacaacaagc aagcaagtga gcaaaactgg gctaattaca gtgcagaaca aaatcgaatg |
| 961 | gggcaggcgg gaagcaccat ctctaactcc catgcacagccttttgattt ccccgatgat |
| 1021 | aaccagaatt ctaaaaaactagctgctgga catgaattac agccactagc cattgtggac |
| 1081 | cagcgacctt caagcagagc cagcagtcgtgccagcagca gacctcggcctgatgacctg |
| 1141 | gagatctag |

Gap Junction Modulation Agents

Certain anti-connexin agents described herein are capable of modulation or affecting the transport of molecules into and out of cells (e.g. blocking or inhibiting). Thus certain gap junction modulation agents described herein modulate cellular communication (e.g. cell to cell). Certain gap junction modulation agents modulate or affect transmission of molecules between the cell cytoplasm and the periplasmic or extracellular space. Such agents are generally targeted to hemichannels (also called connexons), which may be independently involved in the exchange of small molecules between the cell cytoplasm and an extracellular space or tissue. Thus, a compound provided herein may directly or indirectly reduce coupling between cells (via gap junctions) or between a cell and an extracellular space or tissue (via hemichannels), and the modulation of transport of molecules from a cell into an extracellular space is within the scope of certain compounds and embodiments of the invention.

Any molecule that is capable of eliciting a desired inhibition of the passage (e.g. transport) of molecules through a gap junction or hemichannel may be used in embodiments of the invention. Compounds that modulate the passage of molecules through a gap junction or hemichannel are also provided in particular embodiments (e.g., those that modulate the passage of molecules from the cytoplasm of a cell into an extracellular space). Such compounds may modulate the passage of molecules through a gap junction or hemichannel with or without gap junction uncoupling. Such compounds include, for example, binding proteins, polypeptides, and other organic compound that can, for example, block the function or activity of a gap junction or a hemichannel in whole or in part.

As used herein, “gap junction modulation agent” may broadly include those agents or compounds that prevent, decrease or modulate, in whole or in part, the activity, function, or formation of a hemichannel or a gap junction. In certain embodiments, a gap junction modulation agent prevents or decreases, in whole or in part, the function of a hemichannel or a gap junction. In certain embodiments, a gap junction modulation agent induces closure, in whole or in part, of a hemichannel or a gap junction. In other embodiments, a gap junction modulation agent blocks, in whole or in part, a hemichannel or a gap junction. In certain embodiments, a gap junction modulation agent decreases or prevents, in whole or in part, the opening of a hemichannel or gap junction. In certain embodiments, said blocking or closure of a gap junction or hemichannel by a gap junction modulation agent can reduce or inhibit extracellular hemichannel communication by preventing or decreasing the flow of small molecules through an open channel to and from an extracellular or periplamic space. Peptidomimetics, and gap junction phosphorylation compounds that block hemichannel and/or gap junction opening are presently preferred.

In certain embodiments, a gap junction modulation agent prevents, decreases or alters the activity or function of a hemichannel or a gap junction. As used herein, modification of the gap junction activity or function may include the closing of gap junctions, closing of hemichannels, and/or passage of molecules or ions through gap junctions and/or hemichannels.

Exemplary gap junction modulation agents may include, without limitation, polypeptides (e.g. peptiditomimetics, antibodies, binding fragments thereof, and synthetic constructs), and other gap junction blocking agents, and gap junction protein phosphorylating agents. Exemplary compounds used for closing gap junctions (e.g. phosphorylating connexin 43 tyrosine residue) have been reported in U.S. Pat. No. 7,153,822 to Jensen et al., U.S. Pat. No. 7,250,397, and assorted patent publications. Exemplary peptides and peptidomimetics are reported in Green et al., WO2006134494. See also Gourdie et al., see WO2006069181, and Tudor et al., see WO2003032964.

As used herein, “gap junction phosphorylating agent” may include those agents or compounds capable of inducing phosphorylation on connexin amino acid residues in order to induce gap junction or hemichannel closure. Gap junction modulation exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B (2001) J. Biol. Chem., Vol. 276, Issue 11, 8544-8549. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Polypeptide compounds, including binding proteins (e.g. antibodies, antibody fragments, and the like), peptides, peptidomimetics, and peptidomimetics, are suitable modulators of gap junctions.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding protein known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, connexon, gap junctions, or associated molecules).

Binding proteins will generally have a desired specificity, including but not limited to binding specificity, and desired affinity. Affinity, for example, may be a Ka of greater than or equal to about 104 M-1, greater than or equal to about 106 M-1, greater than or equal to about 107 M-1, greater than or equal to about 108 M-1. Affinities of even greater than about 108 M-1 are suitable, such as affinities equal to or greater than about 109 M-1, about 1010 M-1, about 1011 M-1, and about 1012 M-1. Affinities of binding proteins according to the present invention can be readily determined using conventional techniques, for example those described by Scatchard et al., (1949) Ann. N.Y. Acad. Sci. 51: 660.

The invention includes use of peptides (including peptidomimetic and peptidomimetics) for modulation of gap junctions and hemichannels. By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. (1988) J Cell Biol 107: 1817-1824; Meyer R. A. (1992) J Cell Biol 119: 179-189.

Peptides or variants thereof, can be synthesized in vitro, e.g., by the solid phase peptide synthetic method or by enzyme-catalyzed peptide synthesis or with the aid of recombinant DNA technology. Solid phase peptide synthetic method is an established and widely used method, which is described in references such as the following: Stewart et al., (1969) Solid Phase Peptide Synthesis, W. H. Freeman Co., San Francisco; Merrifield, (1963) J. Am. Chem. Soc. 85 2149; Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

The extracellular domains of a hemichannel contributed by two adjacent cells “dock” with each other to form complete gap junction channels. Reagents that interfere with the interactions of these extracellular domains can impair cell-to-cell communication, or with hemichannel opening to the extracellular environment.

Gap junction modulation agents include peptides comprising an amino acid sequence corresponding to a transmembrane region (e.g. 1st to 4th) of a connexin (e.g. connexin 45, 43, 26, 30, 31.1, and 37). Gap junction modulation agents including a peptide comprising an amino acid sequence corresponding to a portion of a transmembrane region of a connexin 43 are preferred for use in the present inventions.

Gap junction modulation agents may comprise a peptide comprising an amino acid sequence corresponding to a portion of a transmembrane region of a connexin 45. Gap junction modulation agents include a peptide having an amino acid sequence that comprises about 5 to 20 contiguous amino acids of SEQ. ID. NO.:13, a peptide having an amino acid sequence that comprises about 8 to 15 contiguous amino acids of SEQ. ID. NO:13, or a peptide having an amino acid sequence that comprises about 11 to 13 contiguous amino acids of SEQ. ID. NO.:13. Other embodiments are directed to an gap junction modulation compound that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO.:13. In certain gap junction modulation compounds provided herein, the extracellular domains of connexin 45 corresponding to the amino acids at positions 46-75 and 199-228 of SEQ ID NO: 13 may be used to develop the particular peptide sequences. Certain peptides described herein have an amino acid sequence corresponding to the regions at positions 46-75 and 199-228 of SEQ. ID. NO: 13. The peptides need not have an amino acid sequence identical to those portions of SEQ. ID. NO: 13, and conservative amino acid changes may be made such that the peptides retain binding activity or functional activity. Alternatively, the peptide may target regions of the connexin protein other than the extracellular domains (e.g. the portions of SEQ. ID. NO:13 not corresponding to positions 46-75 and 199-228).

Also, suitable gap junction modulation agents can include a peptide comprising an amino acid sequence corresponding to a portion of a transmembrane region of a connexin 43. Gap junction modulation agents include peptides having an amino acid sequence that comprises about 5 to 20 contiguous amino acids of SEQ. ID. NO:14, peptides having an amino acid sequence that comprises about 8 to 15 contiguous amino acids of SEQ. ID. NO:14, or peptides having an amino acid sequence that comprises about 11 to 13 contiguous amino acids of SEQ. ID. NO:14. Other gap junction modulation agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:14. Other gap junction modulation agents comprise the extracellular domains of connexin 43 corresponding to the amino acids at positions 37-76 and 178-208 of SEQ. ID. NO:14. Gap junction modulation agents include peptides described herein which have an amino acid sequence corresponding to the regions at positions 37-76 and 178-208 of SEQ. ID. NO:14. The peptides need not have an amino acid sequence identical to those portions of SEQ. ID. NO:14, and conservative amino acid changes may be made such that the peptides retain binding activity or functional activity. Alternatively, peptides may target regions of the connexin protein other than the extracellular domains (e.g. the portions of SEQ. ID. NO:14 not corresponding to positions 37-76 and 178-208).

Still other anti-connexin agents include connexin carboxy-terminal polypeptides. See Gourdie et al., WO2006/069181.

Gap Junction Modifying Agents—Other Anti-Connexin Agents

Gap junction modulation agents, include agents that close or block gap junctions and/or hemichannels or otherwise prevent or decrease cell to cell communication via gap junctions or prevent or decrease cell communication to the extracellular environment via hemichannels. They include agents or compounds that prevent, decrease or inhibit, in whole or in part, the activity, function, or formation of a hemichannel or a gap junction.

In certain embodiments, a gap junction modulation agent induces closure, in whole or in part, of a hemichannel or a gap junction. In other embodiments, a gap junction modifying agent blocks, in whole or in part, a hemichannel or a gap junction. In certain embodiments, a gap junction modifying agent decreases or prevents, in whole or in part, the opening of a hemichannel or gap junction.

In certain embodiments, said blocking or closure of a gap junction or hemichannel by a gap junction modifying agent can reduce or inhibit extracellular hemichannel communication by preventing or decreasing the flow of small molecules through an open channel to and from an extracellular or periplasmic space.

Gap junction modifying agents used for closing hemichannels or gap junctions (e.g. phosphorylating connexin 43 tyrosine residues) have been reported in U.S. Pat. No. 7,153,822 to Jensen et al., U.S. Pat. No. 7,250,397, and assorted patent publications. See also Gourdie et al., see WO2006069181, with regard to connexin carboxy-terminal polypeptides that are said to, for example, inhibit ZO-1 protein binding. Gourdie et al, WO2006069181 describes use of formulations comprising such peptides.

As used herein, “gap junction phosphorylating agent” may include those agents or compounds capable of inducing phosphorylation on connexin amino acid residues in order to induce gap junction or hemichannel closure. Exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B, J. Biol. Chem., Vol. 276, Issue 11, 8544-8549, Mar. 16, 2001. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Still other anti-connexin agents include connexin carboxy-terminal polypeptides. See Gourdie et al., WO2006/069181.

In certain another aspect, gap junction modifying agent may include, for example, aliphatic alcohols; octanol; heptanol; anesthetics (e.g. halothane), ethrane, fluothane, propofol and thiopental; anandamide; arylaminobenzoate (FFA: flufenamic acid and similar derivatives that are lipophilic); carbenoxolone; Chalcone: (2′,5′-dihydroxychalcone); CHFs (Chlorohydroxyfuranones); CMCF (3-chloro-4-(chloromethyl)-5-hydroxy-2(5H)-furanone); dexamethasone; doxorubicin (and other anthraquinone derivatives); eicosanoid thromboxane A(2) (TXA(2)) mimetics; NO (nitric oxide); Fatty acids (e.g. arachidonic acid, oleic acid and lipoxygenase metabolites; Fenamates (flufenamic (FFA), niflumic (NFA) and meclofenamic acids (MFA)); Genistein; glycyrrhetinic acid (GA):18a-glycyrrhetinic acid and 18-beta-glycyrrhetinic acid, and derivatives thereof; lindane; lysophosphatidic acid; mefloquine; menadione; 2-Methyl-1,4-naphthoquinone, vitamin K(3); nafenopin; okadaic acid; oleamide; oleic acid; PH, gating by intracellular acidification; e.g. acidifying agents; polyunsaturated fatty acids; fatty acid WIC inhibitors (e.g. oleic and arachidonic acids); quinidine; quinine; all trans-retinoic acid; and tamoxifen.

### Dosage Forms and Formulations and Administration

A therapeutically effective amount of each of the combination partners (e.g. an anti-connexin polynucleotide and an anti-connexin peptide or peptidomimetic) may be administered simultaneously, separately or sequentially and in any order. The agents may be administered separately or as a fixed combination. When not administered as a fixed combination, preferred methods include the sequential administration of one or more anti-connexin polynucleotides and one or more anti-connexin peptides or peptidomimetics, either or both of which are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered with about one hour of each other, with about one day to about one week of each other, or as otherwise deemed appropriate. Preferably, an anti-connexin peptide or anti-connexin peptidomimetic, e.g., an anti-connexin agent that can block or reduce hemichannel opening, is administered prior to the administration of an anti-connexin agent that blocks or reduce connexin expression or the formation of hemichannels or gap junctions, e.g., by downregulation of connexin protein expression. Preferably, the anti-connexin agent or agents is/are anti-connexin 43 agent(s).

The agents of the invention of the may be administered to a subject in need of treatment, such as a subject with any of the diseases or conditions mentioned herein. The condition of the subject can thus be improved. The anti-connexin agents may thus be used in the treatment of the subject's body by therapy. They may be used in the manufacture of a medicament to treat any of the conditions mentioned herein. Thus, in accordance with the invention, there are provided formulations by which cell-cell communication can be downregulated in a transient and site-specific manner.

The anti-connexin agent may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, e.g. at least about 95%, at least about 98% or at least about 99% of the polynucleotide (or other anti-connexin agent) or dry mass of the preparation.

Depending on the intended route of administration, the pharmaceutical products, pharmaceutical compositions, combined preparations and medicaments of the invention may, for example, take the form of solutions, suspensions, instillations, salves, creams, gels, foams, ointments, emulsions, lotions, paints, sustained release formulations, or powders, and typically contain about 0.1%-95% of active ingredient(s), preferably about 0.2%-70%. Other suitable formulations include pluronic gel-based formulations, carboxymethylcellulose (CMC)-based formulations, and hyroxypropylmethylcellulose (HPMC)-based formulations. Suitable formulations including pluronic gel, have for example about 10 to about 15 percent, suitably about 12 percent, pluronic gel. Other useful formulations include slow or delayed release preparations.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Preferably, the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the agent to the site of application or immediately adjacent that site.

An auxiliary agent such as casein, gelatin, albumin, glue, sodium alginate, carboxymethylcellulose, methylcellulose, hydroxyethylcellulose or polyvinyl alcohol may also be included in the formulation of the invention.

Other suitable formulations include pluronic gel-based formulations, carboxymethylcellulose (CMC)-based formulations, and hyroxypropylmethylcellulose (HPMC)-based formulations. The composition may be formulated for any desired form of delivery, including topical, instillation, parenteral, intramuscular, subcutaneous, or transdermal administration. Other useful formulations include slow or delayed release preparations.

Where the anti-connexin agent is a nucleic acid, such as a polynucleotide, uptake of nucleic acids by mammalian cells is enhanced by several known transfection techniques for example those including the use of transfection agents. Such techniques may be used with certain anti-connexin agents, including polynucleotides. The formulation which is administered may contain such transfection agents. Examples of these agents include cationic agents (for example calcium phosphate and DEAE-dextran) and lipofectants (for example Lipofectam™ and Transfectam™), and surfactants.

Where the anti-connexin agent comprises a polynucleotide, conveniently, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included.

The effective dose for a given subject or condition can be determined by routine experimentation or other methods known in the art or later developed. For example, in order to formulate a range of dosage values, cell culture assays and animal studies can be used. The dosage of such compounds preferably lies within the dose that is therapeutically effective for at least 50% of the population, and that exhibits little or no toxicity at this level.

The effective dosage of each of the anti-connexin agents employed in the methods and compositions of the invention may vary depending on a number of factors including the particular anti-connexin agent or agents employed, the combinational partner, the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

The dose at which an anti-connexin agent is administered to a patient will depend upon a variety of factors such as the age, weight and general condition of the patient, the condition that is being treated, and the particular anti-connexin agent that is being administered.

A suitable therapeutically effective dose of an anti-connexin agent may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight.

Therapeutically effective doses of anti-connexin agents from about 1 to 100, 100-200, 100- or 200-300, 100- or 200- or 300-400, and 100- or 200- or 300- or 400-500 micrograms are appropriate. Doses from about 1-1000 micrograms are also appropriate. Doses up to 2 milligrams may also be used. Doses are adjusted appropriately when the anti-connexin agent or agents are provided in the form of a dressing, typically upward to maintain the desired total dose administration.

Alternatively, in the case of anti-connexin oligonucleotides or anti-connexin peptidomimetics, the dosage of each of the gap junction modulation agents in the compositions may be determined by reference to the composition's concentration relative to the size, length, depth, area or volume of the area to which it will be applied. For example, in certain topical applications, dosing of the pharmaceutical compositions may be calculated based on mass (e.g. grams) of or the concentration in a pharmaceutical composition (e.g. μg/ul) per length, depth, area, or volume of the area of application. Useful doses range from about 1 to about 10 micrograms per square centimeter of wound size. Certain doses will be about 1-2, about 1-5, about 2-4, about 5-7, and about 8-10 micrograms per square centimeter of wound size. Other useful doses are greater than about 10 micrograms per square centimeter of wound size, including at least about 15 micrograms per square centimeter of wound size, at least about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Other doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter.

In certain embodiments, the anti-connexin agent composition may be applied at about 0.01 micromolar (μM) or 0.05 μM to about 200 μM, or up to 300 μM or up to 1000 μM or up to 2000 μM or up to 3200 μM or more final concentration at the treatment site and/or adjacent to the treatment site, and any doses and dose ranges within these dose numbers. Preferably, the antisense polynucleotide composition is applied at about 0.05 μM to about 100 μM final concentration, more preferably, the anti-connexin agent composition is applied at about 1.0 μM to about 50 μM final concentration, and more preferably, the anti-connexin agent composition is applied at about 5-10 μM to about 30-50 μM final concentration. Additionally, the combined anti-connexin agent composition is applied at about 8 μM to about 20 μM final concentration, and alternatively the anti-connexin agent composition is applied at about 10 μM to about 20 μM final concentration, or at about 10 to about 15 μM final concentration. In certain other embodiments, the anti-connexin agent is applied at about 10 μM final concentration. In yet another embodiment, the anti-connexin agent composition is applied at about 1-15 μM final concentration. In other embodiments, the anti-connexin agent is applied at about a 20 μM, 30 μM, 40 μM, 50 μM, 60 μM, 70 μM, 80 μM, 90 μM, 100 μM., 10-200 μM, 200-300 μM, 300-400 μM, 400-500 μM, 500-600 μM, 600-700 μM, 700-800 μM, 800-900 μM, 900-1000 or 1000-1500 μM, or 1500 μM 2000 μM or 2000 μM-3000 μM or greater.

Anti-connexin agent dose amounts include, for example, about 0.1-1, 1-2, 2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL. Milliliter doses are also appropriate for larger treatment sites.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one anti-connexin agent is used, the dosage of each anti-connexin agent need not be in the same range as the other. For example, the dosage of one anti-connexin agent may be between about 0.01 mg to about 10 mg per kg body weight, and the dosage of another anti-connexin agent may be between about 0.1 mg to about 1 mg per kg body weight.

All doses and dose ranges referenced herein are applicable, for example, to anti-connexin oligonucleotides. These dose ranges are also applicable, for example, to anti-connexin peptides anti-connexin mimetic peptides and anti-connexin peptidomimetics.

Conveniently, the anti-connexin agent is administered in a sufficient amount to downregulate expression of a connexin protein, or modulate gap junction formation or connexon opening for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of each of the anti-connexin agents in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. μg/ul) per length, depth, area, or volume of the area of application.

As noted herein, the doses of an anti-connexin polynucleotide, peptide or peptidomimetic administered in combination, or other anti-connexin agents administered in combination with either or both, can be adjusted down from the doses administered when given alone.

The combined use of several agents may reduce the required dosage for any individual agent because the onset and duration of effect of the different agents may be complementary. In a preferred embodiment, the combined use of two or more anti-connexin agents has an additive, synergistic or super-additive effect.

In some cases, the combination of one or more anti-connexin polynucleotide and one or more anti-connexin peptides or peptidomimetics, or other anti-connexin agents administered in combination with either or both, have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of one or more anti-connexin polynucleotides and one or more anti-connexin peptides or peptidomimetics, or other anti-connexin agents administered in combination with either or both, is statistically significantly higher than the sum of the wound healing produced by the individual administration of either of the agents alone. Whether produced by combination administration of one or more anti-connexin polynucleotides and one or more anti-connexin peptides or peptidomimetics, or other anti-connexin agents administered in combination with either or both, is “statistically significantly higher” than the expected additive value of the individual compounds may be determined by a variety of statistical methods as described herein and/or known by one of ordinary skill in the art. The term “synergistic” refers to a type of supra-additive inhibition in which both the anti-connexin polynucleotide and anti-connexin peptide or peptidomimetic, or other anti-connexin agents administered in combination with either or both, individually have the ability to promote wound healing. The term “potentiated” refers to type of supra-additive effect in which one of the anti-connexin polynucleotide, anti-connexin peptides or peptidomimetics, or other anti-connexin agents administered in combination with either or both, individually has the increased ability to promote wound healing.

In general, potentiation may be assessed by determining whether the combination treatment produces a mean wound healing increase in a treatment group that is statistically significantly supra-additive when compared to the sum of the mean wound healing increases produced by the individual treatments in their treatment groups respectively. The mean wound healing increase may be calculated as the difference between control group and treatment group mean wound healing. The fractional increase in wound healing, “fraction affected” (Fa), may be calculated by dividing the treatment group mean wound healing increase by control group mean wound healing. Testing for statistically significant potentiation requires the calculation of Fa for each treatment group. The expected additive Fa for a combination treatment may be taken to be the sum of mean Fas from groups receiving either element of the combination. The Two-Tailed One-Sample T-Test, for example, may be used to evaluate how likely it is that the result obtained by the experiment is due to chance alone, as measured by the p-value. A p-value of less than 0.05 is considered statistically significant, that is, not likely to be due to chance alone. Thus, Fa for the combination treatment group must be statistically significantly higher than the expected additive Fa for the single element treatment groups to deem the combination as resulting in a potentiated supra-additive effect.

Whether a synergistic effect results from a combination treatment may be evaluated by the median-effect/combination-index isobologram method (Chou, T., and Talalay, P. (1984) Ad. Enzyme Reg. 22:27-55). In this method, combination index (CI) values are calculated for different dose-effect levels based on parameters derived from median-effect plots of the anti-connexin agent alone, the one or more agents useful for wound healing alone, and the combination of the two at fixed molar ratios. CI values of & lt; 1 indicate synergy, CI-1 indicates an additive effect, and CP1 indicates an antagonistic effect. This analysis may be performed using computer software tools, such as CalcuSyn, Windows Software for Dose Effect Analysis (Biosoft (D, Cambridge UK).

Any method known or later developed in the art for analyzing whether a supra-additive effect exists for a combination therapy is contemplated for use in screening for suitable anti-connexin agents for use in combination.

In another preferred embodiment, the combined use of one or more anti-connexin polynucleotides and one or more anti-connexin peptides or peptidomimetics reduces the effective dose of any such agent compared to the effective dose when said agent administered alone. In certain embodiments, the effective dose of the agent when used in combination is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-connexin polynucleotides and one or more anti-connexin peptides or peptidomimetics, or other anti-connexin agents in combination with either or both, reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated. Typically, application will be repeated weekly until wound healing is promoted, or a repeat application may be made in the event that wound healing slows or is stalled. Doses may be applied 3-7 days apart, or more. In the case of a chronic wound, repeat applications may be made, for example, weekly, or bi-weekly, or monthly or in other frequency for example if and when wound healing slows or is stalled. For some indications, such as certain ocular uses, more frequent dosing, up to hourly may employed.

One or more anti-connexin polynucleotides and one or more anti-connexin peptides or peptidomimetics may be administered by the same or different routes. The various agents of the invention can be administered separately at different times during the course of therapy, or concurrently in divided or single combination forms.

In one aspect of the invention the anti-connexin polynucleotide is administered in one composition and the anti-connexin peptide or peptidomimetic is administered in a second composition. In one embodiment the first composition comprising one or more anti-connexin peptide or peptidomimetics is administered before the second composition comprising one or more anti-connexin polynucleotides. In one embodiment the first composition comprising one or more anti-connexin peptides or peptidomimetics is administered after the second composition comprising one or more anti-connexin polynucleotides. In one embodiment the first composition comprising one or more anti-connexin peptides or peptidomimetics is administered before and after the second composition comprising one or more anti-connexin polynucleotides. In one embodiment the second composition comprising one or more anti-connexin polynucleotides is administered before and after the first composition comprising one or more anti-connexin peptides or peptidomimetics. In one embodiment the first composition comprising one or more anti-connexin peptides or peptidomimetics is administered about the same time as the second composition comprising one or more anti-connexin polynucleotides.

Preferably one or more anti-connexin polynucleotides and one or more anti-connexin peptides or peptidomimetics, or other anti-connexin agents administered in combination with either or both, are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-connexin polynucleotides and one or more anti-connexin peptides or peptidomimetics, or other anti-connexin agents to be administered in combination with either or both, is dispersed in a slow release solid matrix such as a matrix of alginate, collagen, or a synthetic bioabsorbable polymer. Preferably, the solid support composition is sterile or low bio-burden. In one embodiment, a wash solution comprising two or more anti-connexin agents can be used.

The delivery of a formulation comprising one or more anti-connexin polynucleotides and one or more anti-connexin peptides or peptidomimetics, or other anti-connexin agents to be administered in combination with either or both, over a period of time, in some instances for about 1-2 hours, about 2-4 hours, about 4-6 hours, about 6-8, or about 24 hours or longer, may be a particular advantage in more severe injuries or conditions. In some instances, cell loss may extend well beyond the site of a procedure to surrounding cells. Such loss may occur within 24 hours of the original procedure and is mediated by gap junction cell-cell communication, or hemichannel opening. Administration of anti-connexin agent(s), e.g., for downregulation of connexin expression, or blockade or inhibition of connexon opening or activity, therefore will modulate communication between the cells, or loss into the extracellular space in the case of connexon regulation, and minimize additional cell loss or injury or consequences of injury.

While the delivery period will be dependent upon both the site at which the downregulation is to be induced and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of one or more anti-connexin polynucleotides and one or more anti-connexin peptides or peptidomimetics, or other anti-connexin agents in combination with either or both, in a formulation together with a pharmaceutically acceptable carrier or vehicle, particularly in the form of a formulation for continuous or slow-release administration.

As noted, the one or more agents of the invention may be administered before, during, immediately following wounding, for example, or within about 180, about 120, about 90, about 60, or about 30 days, but preferably within about 10, about 9, about 8, about 7, about 6, about 5, about 4, about 3, or about 2 days or less, and most preferably within about 24, about 12, about 10, about 9, about 8, about 7, about 6, about 5, about 4, about 3, about 2 hours or within about 60, about 45, about 30, about 15, about 10, about 5, about 4, about 3, about 2, about 1 minute following wounding, for example.

The routes of administration and dosages described herein are intended only as a guide since a skilled physician will determine the optimum route of administration and dosage for any particular patient and condition.

Any of the methods of treating a subject having a wound and/or condition referenced or described herein may utilize the administration of any of the doses, dosage forms, formulations, and/or compositions herein described.

Dressings and Matrices

In one aspect, one or more anti-connexin polynucleotides and/or one or more anti-connexin peptides or peptidomimetics are provided in the form of a dressing or matrix. In certain embodiments, the one or more agents of the invention are provided in the form of a liquid, semi solid or solid composition for application directly, or the composition is applied to the surface of, or incorporated into, a solid contacting layer such as a dressing gauze or matrix. The dressing composition may be provided for example, in the form of a fluid or a gel. One or more anti-connexin polynucleotides and one or more anti-connexin peptides or peptidomimetics may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In addition to the biological matrices previously mentioned, suitable dressings or matrices may include, for example, the following with one or more anti-connexin polynucleotides and one or more anti-connexin peptides or peptidomimetics (or other anti-connexin agents to be administered in combination with either or both):

1) Absorptives: suitable absorptives may include, for example, absorptive dressings, which can provide, for example, a semi-adherent quality or a non-adherent layer, combined with highly absorptive layers of fibers, such as for example, cellulose, cotton or rayon. Alternatively, absorptives may be used as a primary or secondary dressing.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source, e.g., pigs or cows. In certain embodiments, a gel or solution is applied to the treatment site and covered with a dressing for barrier protection. In another embodiment, a biological-based (e.g., pig intestinal mucosa or bladder tissue) or biosynthetic-based sheet is placed in situ which may act as membrane, remaining in place after a single application, or the may be biological dressings or biosynthetic dressings may be prepared in advance to include one or more, preferably two, anti-connexin agents.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with treatment site exudate to form a gel. In certain embodiments, collagen dressing may be used in combination with a secondary dressing.

6) Composites: suitable composite dressings may include, for example, dressings that combine physically distinct components into a single product to provide multiple functions, such as, for example, a bacterial barrier, absorption and adhesion. In certain embodiment, the composite dressings are comprised of, for example, multiple layers and incorporate a semi- or non-adherent pad. In certain embodiment, the composite may also include for example, an adhesive border of non-woven fabric tape or transparent film. In certain other embodiment, the composite dressing may function as for example, either a primary or a secondary dressing and in yet another embodiment, the dressing may be used in combination with topical pharmaceutical composition.

7) Contact Layers: suitable contact layer dressings may include, for example, thin, non-adherent sheets placed on an area to protect tissue from for example, direct contact with other agents or dressings applied to the treatment site. In certain embodiments, contact layers may be deployed to conform to the shape of the area of the treatment site and are porous to allow exudate to pass through for absorption by an overlying, secondary dressing. In yet another embodiment, the contact layer dressing may be used in combination with topical pharmaceutical composition.

8) Elastic Bandages: suitable elastic bandages may include, for example, dressings that stretch and conform to the body contours. In certain embodiment, the fabric composition may include for example, cotton, polyester, rayon or nylon. In certain other embodiments, the elastic bandage may for example, provide absorption as a second layer or dressing, to hold a cover in place, to apply pressure or to cushion a treatment site.

9) Foams: suitable foam dressings may include, for example, sheets and other shapes of foamed polymer solutions (including polyurethane) with small, open cells capable of holding fluids. Exemplary foams may be for example, impregnated or layered in combination with other materials. In certain embodiment, the absorption capability may be adjusted based on the thickness and composition of the foam. In certain other embodiments, the area in contact with the treatment site may be non-adhesive for easy removal. In yet another embodiment, the foam may be used in combination with an adhesive border and/or a transparent film coating that can serve as an anti-infective barrier.

10) Gauzes & Non-Woven dressings: suitable gauze dressings and woven dressings may include, for example, dry woven or non-woven sponges and wraps with varying degrees of absorbency. Exemplary fabric composition may include, for example, cotton, polyester or rayon. In certain embodiment, gauzes and non-woven dressing may be available sterile or non-sterile in bulk and with or without an adhesive border. Exemplary gauze dressings and woven dressings may be used for cleansing, packing and covering a variety of treatment sites.

11) Hydrocolloids: suitable hydrocolloid dressings may include, for example, wafers, powders or pastes composed of gelatin, pectin or carboxymethylcellulose. In certain embodiment, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable amorphous hydrogel dressings may include, for example, formulations of water, polymers and other ingredients with no shape, designed to donate moisture and to maintain a moist healing environments and or to rehydrate the treatment site. In certain embodiment, hydrogels may be used in combination with a secondary dressing cover.

13) Hydrogels: Impregnated Dressings: suitable impregnated hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry treatment site and to maintain a moist healing environment.

14) Hydrogel Sheets: suitable hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the treatment site or treated for easy removal.

15) Impregnated Dressings: suitable impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform and scarlet red as well as the compounds described herein.

16) Silicone Gel Sheets: suitable silicone gel sheet dressings may include, for example, soft covers composed of cross-linked polymers reinforced with or bonded to mesh or fabric.

17) Solutions: suitable liquid dressings may include, for example, mixtures of multiprotein material and other elements found in the extracellular matrix. In certain embodiment, exemplary solutions may be applied to the treatment site after debridement and cleansing and then covered with an absorbent dressing or a nonadherent pad.

18) Transparent Films: suitable transparent film dressings may include polymer membranes of varying thickness coated on one side with an adhesive. In certain embodiments, transparent films are impermeable to liquid, water and bacteria but permeable to moisture vapor and atmospheric gases. In certain embodiments, the transparency allows visualization of the treatment site.

Fillers: suitable filler dressings may include, for example, beads, creams, foams, gels, ointments, pads, pastes, pillows, powders, strands or other formulations. In certain embodiment, fillers are non-adherent and may include a time-released antimicrobial. Exemplary fillers may be useful to maintain a moist environment, manage exudate, and for treatment of for example, partial- and full-thickness wounds, infected wounds, draining wounds and deep wounds that require packing.

### Combination Wound Treatment

General Aspects

The present invention is directed to pharmaceutical compositions and their methods of use wherein the composition comprises therapeutically effective amounts of one or more anti-connexin polynucleotides and one or more anti-connexin peptides or peptidomimetics, or other anti-connexin agents in combination with one or more of an anti-connexin polynucleotide and/or an anti-connexin peptide or peptidomimetic. The compositions are useful in enhancing or promoting healing of wounds, including acute wounds and wounds that do not heal at expected rates, such as chronic wounds and other wounds that may be slow to heal or refractory to conventional wound treatment or wound healing promoting therapies.

Equally, in instances of other tissue damage (particularly wounds) the methods and compositions of the invention are effective in promoting the wound healing process, reducing swelling and inflammation, and in minimizing scar formation. The formulations have clear benefit in the treatment of wounds, whether the result of external trauma (including burns), internal trauma, or surgical intervention, as well as chronic wounds.

Compositions

Accordingly, in one aspect, the invention provides compositions for use in therapeutic treatment, which comprises: at least one anti-connexin polynucleotide and at least one anti-connexin peptide or peptidomimetic, or other anti-connexin agents to be administered in combination with either or both or alone. In a preferred embodiment, the composition further comprises a pharmaceutically acceptable carrier or vehicle.

In one preferred form, the composition contains one or more antisense polynucleotides to the mRNA of one connexin protein only. In another preferred form, the composition comprises one or more anti-connexin peptides or peptidomimetics, or a gap junction or hemichannel phosphorylation agent or connexin carboxy-terminal polypeptide. Most preferably, this connexin protein is connexin 43.

In another preferred form, the composition comprises an anti-connexin peptide or pepidomimetic and an antisense polynucleotide to the mRNA of a connexin protein. Most preferably, this connexin is connexin 43.

The compositions may comprise polynucleotides or anti-connexin peptides, or other anti-connexin agents with either or both, that are directed to more than one connexin protein. Preferably, one of the connexin proteins to which polynucleotides or anti-connexin peptides or other anti-connexin agents are directed is connexin 43. Other connexins to which the polynucleotides or anti-connexin peptides or other anti-connexin agents are directed may include, for example, connexins 26, 30, 30.3, 31.1, 32, 36, 37, 40, 40.1, 44.6, 45 and 46. Suitable exemplary polynucleotides (and ODNs) directed to various connexins are set forth in Table 1. Suitable anti-connexin peptides are also provided herein. Suitable gap junction or hemichannel phosphorylation agents and connexin carboxy-terminal polypeptides are known in the art.

Kits, Medicaments and Articles of Manufacture

Optionally, one or more anti-connexin polynucleotides and one or more anti-connexin peptides or peptidomimetics and/or other anti-connexin agents, such as a gap junction or hemichannel phosphorylation agent or connexin carboxy-terminal polypeptide, may also be used in the manufacture of the medicament.

In one aspect, the invention provides a kit comprising one or more compositions or formulations described. For example, the kit may include a composition comprising an effective amount of one or more anti-connexin polynucleotides and one or more anti-connexin peptides or peptidomimetics and/or other anti-connexin agents, such as a gap junction or hemichannel phosphorylation agent or connexin carboxy-terminal polypeptide.

Articles of manufacturer are also provided, comprising a vessel containing a composition or formulation of the invention as described herein and instructions for use for the treatment of a subject. For example, in another aspect, the invention includes an article of manufacture comprising a vessel containing a therapeutically effective amount of one or more anti-connexin polynucleotides and one or more anti-connexin peptides or peptidomimetics and/or other anti-connexin agents, such as a gap junction or hemichannel phosphorylation agent or connexin carboxy-terminal polypeptide, and instructions for use, including use for the treatment of a subject.

Treatment

The compositions and formulations of the invention may be used in conjunction or combination with a composition for promoting the healing of wounds, and can also reduce swelling, inflammation and/or scarring. The compositions and formulations of the invention may also be used in conjunction or combination with a composition for promoting and/or improving the healing of acute or chronic wounds. In one aspect, the wound will be the result of surgery or trauma or underlying medical condition, e.g., diabetes, peripheral edema, vasculitis, or cardiovascular disease.

In one aspect the invention is directed to a method of promoting or improving wound healing in a subject, comprising administration a therapeutically effective amount of one or more anti-connexin polynucleotides and one or more anti-connexin peptides or peptidomimetics or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide. In certain embodiments, the administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, is effective to reduce inflammation, promote cell migration to accelerate wound closure and healing, and/or to facilitate epithelial growth and surface recovery. In certain embodiments, the administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, is effective to reduce or prevent scar formation.

In one aspect the invention is directed to a method of promoting or improving wound healing in a subject, comprising administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, in an amount effective to regulate epithelial basal cell division and growth. In one embodiment, the anti-connexin agent is a connexin antisense polynucleotide effective to regulate epithelial basal cell division and growth. In one embodiment, the connexin antisense polynucleotide is a connexin 26 antisense polynucleotide, peptide or peptidomimetic, a connexin 43 antisense polynucleotide, peptide, or peptidomimetic or a mixture thereof.

In one aspect the invention is directed to a method of promoting or improving wound healing, comprising administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, in an amount effective to regulate outer layer keratin secretion. In one embodiment, the anti-connexin agent is a connexin antisense polynucleotide effective to regulate outer layer keratin secretion. In one embodiment, the connexin antisense polynucleotide is a connexin 43 antisense polynucleotide, peptide or peptidomimetic, a 31.1 antisense polynucleotide, peptide or peptidomimetic or a mixture thereof.

In yet a further aspect, the invention provides a method of decreasing scar formation and/or improving scar appearance in a patient who has suffered a wound, e.g., a surgical wound (such as in, for example, cosmetic and other surgeries), which comprises the step of administering one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to said wound to downregulate expression of one or more connexin protein(s) at and immediately adjacent the site of said wound. Again, the wound may be the result of trauma or surgery, for example, with the formulation being applied to the wound immediately prior to surgical repair and/or closure thereof. As noted herein, in methods to reduce or improve scar formation or appearance, the anti-connexin peptide or peptidomimetic is preferably administered in combination with, or after or prior to, administration of a suitable amount anti-connexin polynucleotide.

In one aspect the invention is directed to a method of reducing, preventing or ameliorating tissue damage in a subject, comprising administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide.

In a further aspect, the invention is directed to a method of reducing swelling and/or inflammation as part of treating an acute or chronic wound and/or tissue subjected to physical trauma which comprises the step of administering one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to or proximate to said wound or tissue. In one embodiment the wound is the result of physical trauma to tissue, including neuronal tissue such as the brain, spinal cord or optic nerve, or skin or eye.

In one aspect the invention is directed to sustained administration of one or more anti-connexin polynucleotides and one or more anti-connexin peptides or peptidomimetics, or, optionally, to sustained administration of one or more anti-connexin polynucleotides and/or one or more anti-connexin peptides or peptidomimetics other anti-connexin agents, such as a gap junction or hemichannel phosphorylation agent or connexin carboxy-terminal polypeptide. In one embodiment, the anti-connexin agents are administered for at least at least about 0.5 hours, about 1-24 hours, at least about 2, hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours or at least about 24 hours. In one embodiment, connexin expression is downregulated over a sustained period of time. In another embodiment, connexin hemichannels are blocked or closed, in whole or in part, over a preferred period of time. Preferably connexin 43 expression is downregulated and connexin hemichannel opening is blocked or inhibited, in whole or in part, for a sustained period of time. Conveniently, connexin 43 expression is downregulated or hemichannels blocked or inhibited for at least about 1, 2, 4, 6, 8, 10, 12, or 24 hours. According to one embodiment, the wound is a chronic wound. Suitable subjects include a diabetic subject. Other subjects include, for example, those with peripheral edema, vasculitis, or cardiovascular disease.

In one aspect, the present invention provides a method of treating a subject having a wound which comprises sustained administration of an effective amount of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to the wound. In a further aspect, the present invention provides a method of promoting or improving wound healing in a subject which comprises sustained administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to a wound. In a further aspect, the present invention provides a method of reducing, preventing or ameliorating swelling and/or inflammation in a subject which comprises sustained administration of one or more anti-connexin polynucleotides and one or more anti-connexin peptides or peptidomimetics, or, optionally, sustained administration of one or more anti-connexin polynucleotides and/or one or more anti-connexin peptides or peptidomimetics other anti-connexin agents, such as a gap junction or hemichannel phosphorylation agent or connexin carboxy-terminal polypeptide, to a wound. In a further aspect, the present invention provides a method of reducing, preventing or ameliorating scar formation in a subject which comprises sustained administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to a wound.

According to another further aspect, the present invention provides a method of promoting or improving wound healing in a subject having a wound which comprises sustained administration of one or more anti-connexin polynucleotides and one or more anti-connexin peptides or peptidomimetics, or, optionally, to sustained administration of one or more anti-connexin polynucleotides and/or one or more anti-connexin peptides or peptidomimetics other anti-connexin agents, such as a gap junction or hemichannel phosphorylation agent or connexin carboxy-terminal polypeptide, to a wound area in an amount effective to increase re-epithlialization rates in the wound area. In one embodiment the method comprises sustained administration of a connexin 43 antisense polynucleotide, peptide and/or peptidomimetic and/or a connexin 31.1 antisense polynucleotide, peptide and/or peptidomimetic. In one embodiment, the composition or compositions are administered in a sustained release formulation. In another embodiment, the composition or compositions are administered for a sustained period of time. Conveniently, the composition is effective to decrease connexin 43 and/or 31.1 levels or activity (e.g., hemichannel or gap junction activity) for at least about 24 hours. According to one embodiment, the wound is a chronic wound. Subjects which may be treated include diabetic subjects.

In yet another aspect, the present invention provides invention provides a method of promoting or improving wound healing in a subject having a wound which comprises sustained administration one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, to a wound area in an amount effective to effective to regulate epithelial basal cell division and growth and/or effective to regulate outer layer keratin secretion. In one embodiment, the composition comprises a connexin antisense polynucleotide effective to regulate epithelial basal cell division or growth, preferably a connexin 26 antisense polynucleotide, peptide and/or peptidomimetic, a connexin 43 antisense polynucleotide, peptide and/or peptidomimetic or a mixture thereof. In one embodiment, the composition comprises a connexin antisense polynucleotide effective to regulate outer layer keratinization, preferably, a connexin 31.1 antisense polynucleotide, peptide and/or peptidomimetic. In one embodiment, the composition or compositions are administered in a sustained release formulation. In another embodiment, the composition or compositions are administered for a sustained period of time. Conveniently, the composition is effective to decrease connexin 43, 26, and/or 31.1 levels for at least about 24 hours. According to one embodiment, the wound is a chronic wound. Subjects which may be treated include diabetic subjects.

In another aspect, methods for treating a subject having a chronic wound are provided. Such methods include administering to the subject an anti-connexin agent capable of inhibiting the expression, formation, or activity of a connexin, or a connexin hemichannel, in combination with another anti-connexin agent.

In one aspect the invention is directed to a method for treatment or prophylaxis of a chronic wound comprising administering to a subject in need thereof an effective amount of an anti-connexin agent administered to said chronic wound or a tissue associated with said chronic wound in combination with another anti-connexin agent. In another embodiment, the chronic wound is a chronic skin wound and a composition of the present invention is administered to the skin or a tissue associated with the skin of said subject for an effective period of time. A chronic skin wound suitable for treatment may, for example, be selected from the group consisting of pressure ulcers, diabetic ulcers, venous ulcers, arterial ulcers, vasculitic ulcers, and mixed ulcers, and other noted herein. The chronic wound may be an arterial ulcer which comprises ulcerations resulting from complete or partial arterial blockage. The chronic wound may be a venous stasis ulcer which comprises ulcerations resulting from a malfunction of the venous valve and the associated vascular disease. The chronic wound may be a trauma-induced ulcer. The chronic wound may be a persistent epithelial defect.

When not administered as a fixed combination, preferred methods include the sequential administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide. Preferably, the agents are administered sequentially within at least about one-half hour of each other. The agents may also be administered with about one hour of each other, with about one day to about one week of each other, or as otherwise deemed appropriate. Preferably, an anti-connexin peptide or anti-connexin peptidomimetic, e.g., an anti-connexin agent that can block or reduce hemichannel opening, is administered prior to the administration of an anti-connexin agent that blocks or reduce connexin expression or the formation of hemichannels or gap junctions, e.g., by downregulation of connexin protein expression. Preferably, the anti-connexin agent or agents is/are anti-connexin 43 agent(s).

In another embodiment for treatment of wounds, including chronic wounds, either or both of the one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

In one embodiment the method for treatment or prophylaxis of a chronic wound comprises sustained administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide. In one embodiment, the composition or compositions are administered in a sustained release formulation. In another embodiment, the composition or compositions are administered for a sustained period of time. Conveniently, the composition is effective to decrease connexin 43 levels, or block or reduce connexin 43 hemichannel opening, for at least about 1-2 hours, about 2-4 hours, about 4-6 hours, about 4-8 hours, about 12 hours, about 18 hours, or about 24 hours. Subjects which may be treated include diabetic subjects, and patients with other ulcers, including venous ulcers and others described herein and known in the art.

The following examples which will be understood to be provided by way of illustration only and not to constitute a limitation on the scope of the invention.

### EXAMPLES

### Example 1

Methods of sequentially administering anti-connexin 43 peptide preparation prepared with the following exemplary sequence: SRPTEKTIFII (SEQ. ID. NO.:19) followed by administration of an anti-connexin 43 polynucleotide preparation prepared with the following exemplary sequences: GTA ATT GCG GCA GGA GGA ATT OTT TCT GTC (connexin 43) (SEQ. ID. NO.:2) and GAC AGA AAC AAT TCC TCC TGC CGC ATT TAC (sense control) (SEQ. ID. NO:136) are evaluated for the efficacy in wound healing in rat diabetic model.

Diabetes is induced in adult Sprague-Dawley rats (350-400 g) by a single intraperitoneal injection containing streptozotocin, 65 mg/kg, in citrate buffer (Shawn H R, Clarke S, Lincoln J. (2003). The effectiveness of treatments of diabetic autonomic neuropathy is not the same in autonomic nerves supplying different organs (Id.). The effectiveness of treatments of diabetic autonomic neuropathy is not the same in autonomic nerves supplying different organs (Id.). The effectiveness of treatments of diabetic autonomic neuropathy is not the same in autonomic nerves supplying different organs (Id.) (N=six diabetic, six control per time point). Most diabetic wound-healing studies are carried out two weeks after diabetes induction and the same time point is used for this wound healing study. However, connexin expression in diabetic rat skin is also examined at eight weeks (N=six diabetic, six control per time point) to confirm that the changes detected at two weeks will remain the same. Normal back skin is excised, cryosectioned, immunostained for connexins, imaged by confocal microscopy and the staining quantified as described in Saitongdee et al. (2000) Effects of hibernation on expression of multiple gap junction connexins in hamster myocardium, Cardiovascular Res. 47, 108-115.

Rats are anaesthetised with halothane and their backs are shaved. Two pairs of 5×5 mm full thickness excision wounds are made. 100-500 micrograms of anti-connexin 43 peptide SRPTEKTIFII (SEQ ID NO: 19) in Pluronic F-127 gel was applied to one wound and control (Pluronic F-127 gel only) applied to the second wound.

10 μM of the connexin 43 oligodeoxynucleotide GTA ATT GCG GCA GGA GGA ATT GTT TCT GTC (SEQ. ID. NO: 2) in Pluronic F-127 gel is applied one wound and control (sense) gel to the other at either within 1 minute, 5 minutes, 10 minutes, 30 minutes, 1 hour or 6 hours.

Tissue is harvested on days 1, 2, 5, 10 and 15 after wounding, and sectioned in preparation for connexin immunohistochemistry or H&E staining (Coutinho P, et al. (2003) Dynamic changes in connexin expression correlate with key events in the wound healing process. Cell Biol. Int. 27:525-541). N=six diabetic, six control rats per time point.

Intercellular communication is assessed by applying a 4% solution of Lucifer Yellow CH (Sigma) in a pledget of gelfoam into a fresh, full thickness skin incision. Dye is allowed to transfer for 5 minutes prior to removal of the gel foam and fixation of the tissue. A 10 kD Kd FITC-dextran that will enter injured cells but not pass through gap junctions is used as a control. Tissues are cryoseqtioned and imaged by confocal microscopy on a Leica SP2UV (Leica, Milton Keynes, UK).

Transferred dyes and connexin immunostaining are examined using the confocal microscope. Optimal gain and offset are set in advance and kept constant during the image acquisition process. A series of single optical section images are taken to generate a montage of the skin from the cut. Digital images (eight bit) are analysed using Image-J software (NIH). To assess dye transfer, a 1500×30 pixel region-of-interest box is placed from the cut edge in the mid-dermis and an image intensity graph across the box is generated. A grey level intensity drop below 50 is taken as the point where Lucifer Yellow had traveled. Similarly, in the epidermis, the distance from the cut to where the Lucifer Yellow signal dropped below 50 is recorded. A minimum of three images are analyzed from each animal. To compare levels of connexin protein, six single optical section images of dermis or epidermis are taken from different sections for each wound. All parameters of laser power, pinhole, gain/offset and objective are kept constant across both control and diabetic groups. Connexin expression is quantified as described in Saitongdee P, et al. (2000), supra. A threshold is set to detect gap-junction plaques with minimal background noise and is then kept constant for all images. The number and size of connexin plaques are recorded for each image and expressed per 100 μm of epidermis or 10000 μm² of dermis. This approach has proved to be much more accurate than Western blot as it generates information on protein expression at the cellular level. Western blots are unable to distinguish between epidermal and dermal cells or detect effects of proximity to the wound edge. Using this approach, connexin levels in keratinocytes in a zone at the wound edge (WE) and in a zone 500 μm away (AD) are able to be quantified either one or two days after wounding. At day five after wounding, an additional zone of the leading edge (LE) of the nascent epidermis is also imaged. Images of H&E staining are taken using a Leica DMLFS microscope with a DC300F digital camera. Measurements for re-epithelialization rate are described in detail in Qiu, C et al. (2003) Targeting connexin43 expression accelerates the rate of wound repair. Curr. Biol. 13:1697-1703. All numerical differences between treatments are tested for significance using the Wilcoxon matched-pairs signed-ranks test as implemented in Statview 5.0.1.

Relative connexin 43 and 26 staining levels in normal and STZ diabetic rat skin at two weeks and eight weeks after induction of diabetes are measured and compared. Graphs are plotted to show the numbers of plaques in the epidermis and dermis. Images of typical connexin 43 and connexin 26 immunostaining in control and diabetic skin at eight weeks are acquired (arrowheads mark the boundary between the epidermis and dermis; scale bar 25 μm). The relative distances that the gap-junction-permeant dye, Lucifer Yellow, traveled in five minutes in the epidermis and dermis of the control and diabetic rats are quantified.

Typically punctate connexin 43 immunostaining is found in the basal layer of the epidermis, and in dermal fibroblasts, hair follicles, blood vessels and appendages. However, in diabetic skin, connexin 43 staining may be significantly reduced in the epidermis, in terms of both size and number of gap junction plaques. Staining for connexin 26 in the upper layers of the epidermis may be similarly reduced in diabetic epidermis.

To assess cell-cell communication in diabetic epidermis and dermis, the extent of transfer of the gap-junction-permeant dye Lucifer Yellow through the tissue in five minutes is examined. Elevated expression of connexin 43 protein and increased communication has been reported in human diabetic fibroblasts (Abdullah K M, et al. (1999) Cell-to-cell communication and expression of gap junctional proteins in human diabetic and nondiabetic skin fibroblasts: effects of basic fibroblast growth factor, Endocrine 10:35-41); and mixed responses of different connexins to diabetes have been noted in the renal system (Zhang J, Hill C E. (2005) Differential connexin expression in preglomerular and postglomerular vasculature: accentuation during diabetes, Kidney Int. 68:1171-1185).

Relative rates of re-epithelialization and responses of connexin 43 and connexin 26 protein levels following injury in control and diabetic epidermis are measured. Staining is quantified by counting plaques at one and two days after wounding in epidermis at the wound edge (WE) and adjacent (AD) epidermis, 500 μm away. On day five, an additional zone at the leading edge (LE) of the nascent epidermis is quantified.

Connexin 43 and connexin 26 staining (green) and nuclear staining (blue) at the epidermal wound edge of control and diabetic skin during the wound-healing process are measured and the processed by image analysis.

To determine the dynamic responses of connexin expression to injury, connexin staining in keratinocytes at the wound edge (WE) and in an adjacent zone 500 μm away (AD) is quantified at one and two days after wounding. At after five days the leading edge (LE) keratinocytes is imaged.

The effect of the possible increase of connexin 43 protein in diabetic WE keratinocytes is assessed by preventing the increase with a connexin 43-specific antisense gel, applied to the wound at the time of injury.

A finding of abnormal upregulation of connexin 43 in the epidermal wound edge in diabetes is significant, and has the potential to affect the process of wound closure in different ways. The formation of communication compartments within the regenerating epidermis has been proposed to play a role in wound healing (Martin P (1997) Wound healing—aiming for perfect skin regeneration, Science 276:75-81; Lampe P D, et al. (1998) Cellular interaction of integrin alpha3beta1 with laminin 5 promotes gap junctional communication. J. Cell Biol. 143:1735-1747; Hodgins M (2004) Connecting wounds with connexins. J. Invest. Dermatol. 122: commentary). Compartmentalization could be effectively brought about in normal conditions by expression of connexin 26 and removal of connexin 43 in leading edge cells, as these connexins do not form junctions with one another. Thus, the delay in wound healing in diabetes could reflect the additional time required for connexin 43 expression to downregulate to a point where such a compartmentalization can occur. Alternatively, the C-tail of connexin 43 is known to interact with cytoskeletal components or with P120ctn/Rho GTPase, so downregulation of connexin 43 could be necessary for changing the motility of keratinocytes at the wound edge, enabling them to migrate and close the wound (Wei C J, et al. (2004) Connexins and cell signaling in development and disease, Annu Rev Cell Dev Biol. 20:811-838).

### Example 2

Wound healing efficacy in a diabetic subject is investigated after sequentially administering an anti-connexin 43 peptide preparation prepared with the following exemplary sequence: SRPTEKTIFII (SEQ. ID. NO:19) followed by administration of anti-connexin 43 polynucleotides preparation prepared with the following exemplary sequences: GTA ATT GCG GCA GGA GGA ATT OTT TCT GTC (connexin 43) (SEQ. ID. NO.:2) and GAC AGA AAC AAT TCC TCC TGC CGC ATT TAC (sense control) (SEQ. ID. NO.:136) in vivo to diabetic male Sprague Dawley rats. In order to quantify the wound healing in a diabetic subject, the tensile strength of the wounds is investigated, with a higher tensile strength reflecting an improvement in wound healing.

The diabetic rat animal model is an established model system for investigating diabetes-associated wounds, which heal poorly (Davidson, Arch. Dermatol. Res. 290: S1-S11). Since diabetes is accompanied by microangiopathy, this animal model is also suitable for investigating arterially determined disturbances in wound healing.

In order to induce the diabetes, rats having a bodyweight of 250-300 g are injected i.p. with a freshly prepared aqueous solution of streptozotocin (Sigma) (50 mg/kg of bodyweight). The blood sugar of the animals is checked 7-9 days after induction, with a blood sugar level value of more than 200 mg/dL confirming the diabetic state. The diabetic rats and the nondiabetic control animals are subsequently anaesthetized with a mixture consisting of 2% O₂ (2 l/min) and 1.25% isofluran. The back is depilated and 2 sites are marked on the back of each animal for subsequent wounding. Incision wounds of 1 cm in length are then made through the wound sites and the wounds are closed with wound clips.

100-500 micrograms of anti-connexin 43 peptide SRPTEKTIFII (SEG ID. NO: 19) in Pluronic F-127 gel was applied to one wound and control (Pluronic F-127 gel only) applied to the second wound. Thereafter, 10 μM of the connexin 43 oligodeoxynucleotide GTA ATE GCG GCA GGA GGA ATT GTT TCT GTC (SEQ. ID. NO: 2) in Pluronic F-127 gel is applied one wound and control (sense) gel to the other at either within 1 minute, 5 minutes, 10 minutes, 30 minutes, 1 hour or 6 hours.

The wound biopsies are taken after 10 days and the tensile strength of the wounds is determined using an Instron tensiometer in accordance with the manufacturer's instructions and standardized to the cross sectional area of the wounds.

Subsequently, the quotient (E/C value) is calculated from the absolute value of the tensile strength of a wound which is treated and the absolute value of the tensile strength of a wound in the same animal which only receive the control preparation. The mean of the E/C values is determined and the changes in tensile strength relative to the treatment are determined.

### Example 3

The method and compositions disclosed herein are used to treat a human subject with a chronic wound (e.g., a vasculitic ulcer).

A human subject with diabetes, or underlying peripheral vascular or arterial disease first is presented for complications arising from a non-closing leg wound. The wound is treated with a suitable dose or doses of an anti-connexin peptide SRPTEKTIFII (SEQ ID NO: 19), e.g., 100-500 micrograms. The wound is subsequently treated with the anti-connexin polynucleotide in SEQ ID NO: 1 within 1 minute, 10 minutes, 30 minutes, 1 hour, 6 hours, 12 hours, or 24 hours of the anti-connexin peptide. 2 mL of 20 μM preparation of SEQ ID NO: 1 in pluronic gel (e.g., total dose ˜200-400 μg) based on a wound size of approximately 7 cm×5 cm (about 35 cm²) with a depth of approximately 3-4 mm is administered (other appropriate dosages to be administered can be readily determined by a skilled practitioner in accordance with the wound size).

The wound site is dressed and covered for a period of 7 days. The wound is uncovered on Day 7 and the wound healing results are assessed.

The treatment outlined above is repeated (in appropriate dosage) as necessary or desired. Following this treatment, the wound and the wound appearance is assessed. The patient is evaluated again at two weeks, one month, and/or two months and the wound (reduction if any) is again evaluated.

### Example 4

The method and compositions disclosed herein are used to treat a human subject with a chronic venous leg ulceration.

Human test subjects are grouped according to ulcer size and minimum and maximum ulcer areas (e.g., 2 cm² and 50 cm²). Patient's resting ankle brachial doppler arterial pressure index are determined as a baseline (e.g. equal to or greater than 0.80).

All patients receive compression bandaging. The area of the ulcer is determined by tracing, and suitable dosages of test anti-connexin peptide SRPTEKTIFII (SEQ ID NO: 19) in a pluronic gel preparation, e.g., 100-500 micrograms, is administered sequentially followed by administration of the anti-connexin polynucleotide in SEQ ID NO: 1 (also in pluronic gel with total dose of approximately 200-400 μg) within 1 minute, 10 minutes, 30 minutes, 1 hour, 6 hours, 12 hours, or 24 hours of the anti-connexin peptide.

The patient is asked to lie recumbent on the examination couch while the test preparation is applied to the surface of the wound, and to remain there for 15 minutes before compression bandaging is applied.

The wound is uncovered on Day 7 and the wound healing results are assessed. Wound fluids and blood samples are analyzed for relevant wound healing biomarkers using bioassays.

The treatment outlined above is repeated (in appropriate dosage). Following this treatment, the wound and the wound appearance is assessed. This course is repeated weekly or bi-weekly, or as appropriate given the state of healing, until wound closure.

### Example 5

The following are used to determine therapeutic efficacy of sequential administration of exemplary preparations in accelerating the healing rate of diabetic and other chronic ulcers.

The primary efficacy endpoint is the percentage of patients achieving full wound closure within 12-20 weeks. Secondary endpoints include the time to 100% closure, time to 80% closure, time to 50% closure, and the amount of wound closure as a percentage change from the baseline wound size at 3, 5, 10, 15, and 20 weeks. Kaplan-Meier survival analysis techniques are utilized to examine the time-to-event endpoints.

All patients receive a regimen of standard diabetic (or other) ulcer care consisting of initial sharp debridement, wound cleansing, wound dressing, and wound pressure offloading. The ulcer is treated by an initial sharp debridement and wound cleansing. 100-500 micrograms of an anti-connexin peptide SRPTEKTIFII (SEQ ID NO: 19) in a pluronic gel preparation is administered sequentially followed by administration of the anti-connexin polynucleotide in SEQ ID NO: 1 (also in Pluronic gel with total dose of approximately 200-400 μg) within 1 minute, 10 minutes, 30 minutes, 1 hour, 6 hours, 12 hours, or 24 hours of the anti-connexin peptide. The wound is dressed with a non-stick bandage and pressure bandage.

Wounds are evaluated twice a week for up to 12-20 weeks or until wound closure, whichever is earlier. Patients are removed from the study if they developed a clinical infection or if the wound condition significantly deteriorated. At each wound evaluation (twice weekly), the wound perimeter is traced for determination of wound area, and the wound is photographed with a digital camera. Blood chemistry and hematology tests are performed at patient enrollment, and at weeks 5, 10, 15, and 20. A radiographic assessment may be conducted every 5 weeks to study effects on underlying bone composition.

### Example 6

Anti-connexin agent is conveniently formulated in a form suitable for administration according to the methods of the present invention.

Suitable formulations include a mixture of the following formulating agents. The amount of the individual anti-connexin agent or agents and formulating agents will depend of the particular use intended.

ASO in PBS
Polyquaternium 10
HEC/HPMC/CMC
Na Hyaluronate
Tween 20
Poloxamer 188
Pluronic 87 NF
SLES
Poly L-lysine/Polyethylene Imine
Benzalkonium chloride
Methyl paraben
Propyl paraben
Propylene Glycol
10 mM Phosphate Buffer

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method of treatment comprising administering to a subject in need thereof a composition comprising therapeutically effective amounts of a first anti-connexin agent and a second anti-connexin agent, wherein said first agent is an anti-connexin polynucleotide agent and said second agent is an anti-connexin peptide or peptidomimetic.

2. A method according to claim 1, wherein said polynucleotide is an antisense polynucleotide.

3. A method according to claim 2, wherein said antisense polynucleotide comprises a sequence selected from SEQ. ID. NOS:1 to 12.

4. A method according to claim 2, wherein said antisense polynucleotide is selected from:
(SEQ ID NO: 1)
GTA ATT GCG GCA AGA AGA ATT GTT TCT GTC;
(SEQ ID NO: 2)
GTA ATT GCG GCA GGA GGA ATT GTT TCT GTC;
and,
(SEQ ID NO: 3)
GGC AAG AGA CAC CAA AGA CAC TAC CAG CAT.

5. A method according to claim 2, wherein said antisense polynucleotide has from about 15 to about 35 nucleotides and is sufficiently complementary to connexin 43 mRNA to form a duplex having a melting point greater than 20° C. under physiological conditions.

6. A method according to claim 2, wherein the antisense polynucleotide has from about 15 to about 35 nucleotides and has at least about 70 percent homology to an antisense sequence of connexin 43 mRNA.

7. A method according to claim 1, wherein the composition comprises about 0.1 to about 1000 micrograms of said anti-connexin agent and the anti-connexin 43 agent is an antisense polynucleotide.

8. A method of claim 1, wherein said peptide comprises a sequence selected from SEQ. ID. NOS:14 to 23.

9. A method according to claim 1, wherein the composition comprises about 0.01 to about 100 milligrams of said anti-connexin 43 peptide or anti-connexin 43 peptidomimetic.

10. A method according to claim 1, wherein said anti-connexin agent is an RNAi or siRNA polynucleotide.

11. A method according to claim 1, wherein the subject is a mammal.

12. A method according to claim 11, wherein the mammal is a human.

13. A method according to claim 11, wherein the mammal is selected from the group consisting of domestic animals, farm animals, zoo animals, sports animals, and pets.

14. A method according to claim 1, wherein the subject has a wound.

15. A method of treatment comprising administering to a subject in need thereof a first composition and a second composition, said first composition comprising a therapeutically effective amount of a anti-connexin 43 polynucleotide and said second composition comprising a therapeutically effective amount of an anti-connexin 43 peptide or peptidomimetic.

16. A method according to claim 15, wherein the first and second compositions are administered simultaneously.

17. A method according to claim 15, wherein the first and second compositions are administered within at least about one-half hour of each other.

18. A method according to claim 15, wherein first and second compositions are administered within about one hour of each other, within about one day of each other, or within about one week of each other.

19. A method according to claim 15, wherein the first composition is administered first.

20. A method according to claim 15, wherein the second composition is administered first.

21. A method according to claim 15, further comprising administration of a third composition, wherein the third wound healing composition comprises an anti-connexin polynucleotide, peptide or peptidomimetic.

22. A method according to claim 15, wherein the third composition is administered first.

23. A method according to claim 15, wherein said polynucleotide is an antisense polynucleotide.

24. A method according to claim 23, wherein said antisense polynucleotide comprises a sequence selected from SEQ. ID. NOS:1 to 12.

25. A method according to claim 23, wherein said antisense polynucleotide is selected from:
(SEQ ID NO: 1)
GTA ATT GCG GCA AGA AGA ATT GTT TCT GTC;
(SEQ ID NO: 2)
GTA ATT GCG GCA GGA GGA ATT GTT TCT GTC;
and,
(SEQ ID NO: 3)
GGC AAG AGA CAC CAA AGA CAC TAC CAG CAT.

26. A method according to claim 23, wherein said antisense polynucleotide has from about 15 to about 35 nucleotides and is sufficiently complementary to connexin 43 mRNA to form a duplex having a melting point greater than 20° C. under physiological conditions.

27. A method according to claim 23, wherein the antisense polynucleotide has from about 15 to about 35 nucleotides and has at least about 70 percent homology to an antisense sequence of connexin 43 mRNA.

28. A method according to claim 15, wherein the composition comprises about 0.1 to about 1000 micrograms of said anti-connexin agent and the anti-connexin 43 agent is an antisense polynucleotide.

29. A method of claim 15, wherein said peptide comprises a sequence selected from SEQ. ID. NOS:14 to 23.

30. A method according to claim 15, wherein the composition comprises about 0.01 to about 100 milligrams of said anti-connexin 43 peptide or anti-connexin 43 peptidomimetic.

31. A method according to claim 15, wherein said anti-connexin agent is an RNAi or siRNA polynucleotide.

32. A method according to claim 15, wherein the subject is a mammal.

33. A method according to claim 32, wherein the mammal is a human.

34. A method according to claim 34, wherein the mammal is selected from the group consisting of domestic animals, farm animals, zoo animals, sports animals, and pets.

35. A method according to claim 15, wherein the subject has a wound.

36. A method according to claim 15, wherein the subject has a chronic wound.

37. A method according to claim 36, wherein the chronic wound is a diabetic ulcer.

38. A method according to claim 36, wherein the chronic wound is a venous ulcer.

39. A method according to claim 36, wherein the chronic wound is a pressure ulcer, a vasculitic ulcer, or an arterial ulcer.

40. A pharmaceutical composition for use in promoting or improving wound healing, which comprises therapeutically effective amounts of an anti-connexin 43 polynucleotide and an anti-connexin 43 peptide or peptidomimetic.

41. A pharmaceutical composition according to claim 40, wherein said polynucleotide is an antisense polynucleotide.

42. A pharmaceutical composition according to claim 41, wherein said antisense polynucleotide comprises a sequence selected from SEQ. ID. NOS:1 to 12.

43. A pharmaceutical composition according to claim 41, wherein said antisense polynucleotide is selected from:
(SEQ ID NO: 1)
GTA ATT GCG GCA AGA AGA ATT GTT TCT GTC;
(SEQ ID NO: 2)
GTA ATT GCG GCA GGA GGA ATT GTT TCT GTC;
and,
(SEQ ID NO: 3)
GGC AAG AGA CAC CAA AGA CAC TAC CAG CAT.

44. A pharmaceutical composition according to claim 41, wherein said antisense polynucleotide has from about 15 to about 35 nucleotides and is sufficiently complementary to connexin 43 mRNA to form a duplex having a melting point greater than 20° C. under physiological conditions.

45. A pharmaceutical composition according to claim 41, wherein the antisense polynucleotide has from about 15 to about 35 nucleotides and has at least about 70 percent homology to an antisense sequence of connexin 43 mRNA.

46. A pharmaceutical composition according to claim 41, wherein the composition comprises about 0.1 to about 1000 micrograms of said anti-connexin agent and the anti-connexin 43 agent is an antisense polynucleotide.

47. A pharmaceutical composition of claim 40, wherein said peptide comprises a sequence selected from SEQ. ID. NOS:14 to 23.

48. A pharmaceutical composition according to claim 40, wherein the composition comprises about 0.01 to about 100 milligrams of said anti-connexin 43 peptide or anti-connexin 43 peptidomimetic.

49. A pharmaceutical composition according to claim 40, wherein said anti-connexin agent is an RNAi or siRNA polynucleotide.

50. A pharmaceutical composition according to claim 40 which is formulated for topical administration.

51. A pharmaceutical composition according to claim 40 which is formulated as a gel.

52. A pharmaceutical composition according to claim 40, wherein said gel is a polyoxyethylene-polyoxypropylene copolymer-based gel or a carboxymethylcellulose-based gel.

53. A pharmaceutical composition according to claim 40, wherein said gel is a pluronic gel.

54. A method for treating chronic wounds, comprising administering to a subject in need thereof a therapeutically effective amount of a pharmaceutical composition according to claim 40.

55. A method according to claim 54, wherein the chronic wound is a diabetic ulcer.

56. A method according to claim 54, wherein the chronic wound is a venous ulcer.

57. A method according to claim 54, wherein the chronic wound is a pressure ulcer, a vasculitic ulcer, or an arterial ulcer.

58. A method for reducing scar formation in a subject in need thereof, comprising administering to said subject a therapeutically effective amount of a pharmaceutical composition according claim 40.

59. A method according to claim 58, wherein the pharmaceutical composition is administered topically.

60. A method of preparing a medicament for treating a wound, comprising bringing together and an amount of a first composition and a second composition, wherein said first composition comprises an effective amount of an anti-connexin polynucleotide and said second composition comprises an effective amount of an anti-connexin peptide or peptidomimetic.

61. A method according to claim 60 wherein said anti-connexin agent comprises an anti-connexin 43 antisense polynucleotide.

62. A method of claim 61 wherein said medicament is formulated for topical administration.

63. A method of claim 61 wherein said medicament is formulated for sustained release.

64. An article of manufacture comprising package material containing a pharmaceutical composition according to claim 40 together with instructions for use in or on a subject in order to promote or improve wound healing or tissue repair.

65. A wound dressing comprising an anti-connexin polynucleotide agent and an anti-connexin peptide or peptidomimetic.

66. A method according to claim 15 wherein the wound is a persistent epithelial defect.
